SEQUENCE LISTING (107 sequences)

SEQ ID NO: 1 - protein, 467 aa, synthetic construct
  REGION 1..467: note = Anti-QSOX1 heavy chain H2a
MGWSCIILFLVATATGVHCQITLKESGPTLVKPTQTLTLTCTVSGFSLTGYGVNWIRQPPGKALEWLGMIWGDGRTDYSPSLKSRLTITKDTSKNQVVLTMTNMDPVDTATYYCASDYYGSGSFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 2 - protein, 467 aa, synthetic construct
  REGION 1..467: note = Anti-QSOX1 heavy chain H2b (hQSOX1.2)
MGWSCIILFLVATATGVHCQITLKESGPTLVKPTQTLTLTCTVSGFSLTGYGVNWIRQPPGKALEWLGMIWGDGRTDYSPSLKSRLTITKDTSKNQVVLTMTNMDPVDTATYYCASDYYGSGSFAYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 3 - protein, 467 aa, synthetic construct
  REGION 1..467: note = Anti-QSOX1 heavy chain H3 (h QSOX1.1)
MGWSCIILFLVATATGVHCQVQLVESGGGLVQPGGSLRLSCSVSGFSLTGYGVNWVRQAPGKGLEWLGMIWGDGRTDYADSVKGRFTISRDNSKNTLYLQMSSLRAEDTAVYYCASDYYGSGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 4 - protein, 467 aa, synthetic construct
  REGION 1..467: note = Anti-QSOX1 heavy chain H3new
MGWSCIILFLVATATGVHCQVQLVESGGGLVQPGRSLRLSCTVSGFSLTGYGVNWFRQAPGKGLEWLGMIWGDGRTDYTASVKGRFTISRDGSKSIAYLQMNSLKTEDTAVYYCASDYYGSGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 5 - protein, 233 aa, synthetic construct
  REGION 1..233: note = Anti-QSOX1 light chain K1a
MGWSCIILFLVATATGVHSDIQMTQSPSSLSASVGDRVTITCKASQDVSTAVAWYQQKPGKAPKLLIHSASYRYTGVPSRFSGSGSGTDFTFTISSLQPEDIATYYCQQHYSIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 233 aa, synthetic construct
  REGION 1..233: note = Anti-QSOX1 light chain K2
MGWSCIILFLVATATGVHSDIVMTQTPLSLSVTPGQPASISCKASQDVSTAVAWYLQKPGQPPQLLIHSASYRYTGVPDRFSGSGSGTDFTLKISRVEAEDVGVYYCQQHYSIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 233 aa, synthetic construct
  REGION 1..233: note = Anti-QSOX1 light chain K3
MGWSCIILFLVATATGVHSEIVMTQSPATLSLSPGERATLSCKASQDVSTAVAWYQQKPGQAPRLLIHSASYRYTGIPARFSGSGSGTDFTLTISSLQPEDFAVYYCQQHYSIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 233 aa, synthetic construct
  REGION 1..233: note = Anti-QSOX1 light chain K4 (hQSOX1.1, hQSOX1.2)
MGWSCIILFLVATATGVHSDIVMTQSPDSLAVSLGERATINCKASQDVSTAVAWYQQKPGQPPKLLIHSASYRYTGVPDRFSGSGSGTDFTLTISSLQAEDVAVYYCQQHYSIPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 233 aa, synthetic construct
  REGION 1..233: note = Anti-QSOX1 light chain K1b
MGWSCIILFLVATATGVHSAIQLTQSPSSLSASVGDRVTITCKASQDVSTAVAWYQQKPGKAPKLLIHSASYRYTGVPSRFSGSGSGTDFTLTISSLQPEDFATYYCQQHYSIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 468 aa, synthetic construct
  REGION 1..468: note = Anti-PSA Design 1,2 heavy chain
MDWTWRFLFVVAAATGVQSQVQLVQSGAEVKKPGSSVKVSCKASGYSFTDYNMNWVRQAPGQGLEWIGVISPDYGTPSYAQKFQGRVTITADESTSTAYMELSSLRSEDTAVYYCARGKGNYVVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 234 aa, synthetic construct
  REGION 1..234: note = Anti-PSA Design 1 light chain
MRVPAQLLGLLLLWLSGARCDIQMTQSPSSLSASVGDRVTITCKASQSVVNNVVWYQQKPGKAPKLLIYYASNRYSGVPSRFSGSGSGTDFTLTISSLQPEDFATYYCQQHYTSPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 234 aa, synthetic construct
  REGION 1..234: note = Anti-PSA Design 2 light chain
MRVPAQLLGLLLLWLSGARCDIVMTQTPLSLSVTPGQPASISCKASQSVVNNVVWYLQKPGQPPQLLIYYASNRYSGVPDRFSGSGSGTDFTLKISRVEAEDVGVYYCQQHYTSPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 468 aa, synthetic construct
  REGION 1..468: note = Anti-PSA Design 3,5 heavy chain
MDWTWRFLFVVAAATGVQSEVQLVQSGAEVKKPGATVKISCKVSGYSFTDYNMNWVQQAPGKGLEWIGVISPDYGTPSYAEKFQGRVTITADTSTDTAYMELSSLRSEDTAVYYCARGKGNYVVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 234 aa, synthetic construct
  REGION 1..234: note = Anti-PSA Design 3 light chain
MRVPAQLLGLLLLWLSGARCDIVMTQSPDSLAVSLGERATINCKASQSVVNNVVWYQQKPGQPPKLLIYYASNRYSGVPDRFSGSGSGTDFTLTISSLQAEDVAVYYCQQHYTSPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 468 aa, synthetic construct
  REGION 1..468: note = Anti-PSA Design 4 heavy chain
MDWTWRFLFVVAAATGVQSQVQLQESGPGLVKPSETLSLTCTVSGYSFTDYNMNWIRQPPGKGLEWIGVISPDYGTPSYNPSLKSRVTISVDTSKNQFSLKLSSVTAADTAVYYCARGKGNYVVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 234 aa, synthetic construct
  REGION 1..234: note = Anti-PSA Design 4,5 light chain
MRVPAQLLGLLLLWLSGARCDIQMTQSPSSLSASVGDRVTITCKASQSVVNNVVWYQQKPGKAPKLLIYYASNRYSGVPSRFSGSGSGTEFTLTISSLQPEDFATYYCQQHYTSPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 471 aa, synthetic construct
  REGION 1..471: note = Anti-AXL humanized heavy chain (hAXL)
MDWTWRFLFVVAAATGVQSEVQLVESGGGLIQPGGSLRLSCAASGFTFSTYGMSWVRQAPGKGLELVATINGNGGSAYYADSVKGRFTISRDNSKNTLYLQMNSLRAEDTAVYYCVRERDYYGNSRYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 234 aa, synthetic construct
  REGION 1..234: note = Anti-AXL humanized light chain (hAXL)
MRVPAQLLGLLLLWLSGARCAIQLTQSPSSLSASVGDRVTITCKASQNVDTNVAWYQQKPGKAPKPLIFSAFLRYSGVPSRFSGSGSGTDFTLTISSLQPEDFATYYCQQYNNYPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 247 aa, synthetic construct
  REGION 1..247: note = Anti-HEWL Design 1 heavy chain (hHEWL) Fab
MGWSCIILFLVATATGVHCEVQLVQSGAEVKKPGATVKISCKVSGYTFSTYWMEWVQQAPGKGLEWMGEVLPGSGSTYYAEKFQGRVTITADTSTDTAYMELSSLRSEDTAVYYCARGDGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHHHHHH

SEQ ID NO: 20 - protein, 234 aa, synthetic construct
  REGION 1..234: note = Anti-HEWL Design 1 light chain (hHEWL) Fab
MGWSCIILFLVATATGVHSDDIQMTQSPSSLSASVGDRVTITCRASQSISSNLNWYQQKPGKAPKLLIYYASSLQSGVPSRFSGSGSGTDFTLTISSLQPEDFATYYCQQSNSWPRTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 116 aa, synthetic construct
  REGION 1..116: note = Anti-HEWL Design 2 heavy chain variable
QVQLVQSGAEVKKPGSSVKVSCKASGYTFSTYWIEWVRQAPGQGLEWMGEILPGSGSAYYAQKFQGRVTITADESTSTAYMELSSLRSEDTAVYYCARGDGYFDLWGRGTLVTVSS

SEQ ID NO: 22 - protein, 107 aa, synthetic construct
  REGION 1..107: note = Anti-HEWL Design 1,2 light chain variable
DIQMTQSPSSLSASVGDRVTITCRASQSISSNLNWYQQKPGKAPKLLIYYASSLQSGVPSRFSGSGSGTDFTLTISSLQPEDFATYYCQQSNSWPRTFGPGTKVDIK

SEQ ID NO: 23 - protein, 116 aa, synthetic construct
  REGION 1..116: note = Anti-HEWL Design 3 heavy chain variable
EVQLVQSGAEVKKPGATVKISCKVSGYTFSTYWMEWVQQAPGKGLEWMGEVLPGSGSTYYAEKFQGRVTITADTSTDTAYMELSSLRSEDTAVYYCARGDGYFDYWGQGTLVTVSS

SEQ ID NO: 24 - protein, 107 aa, synthetic construct
  REGION 1..107: note = Anti-HEWL Design 3 light chain variable
AIQMTQSPSSLSASVGDRVTITCRASQGISNNLGWYQQKPGKAPKLLIYYASSLQSGVPSRFSGSGSGTDFTLTISSLQPEDFATYYCLQSNSWPRTFGPGTKVDIK

SEQ ID NO: 25 - protein, 116 aa, synthetic construct
  REGION 1..116: note = Anti-HEWL Design 4 heavy chain variable
QVQLVQSGAEVKKPGSSVKVSCKASGYTFSTYWIEWVRQAPGQGLEWMGEILPGSGSAYYAQKFQGRVTITADESTSTAYMELSSLRSEDTAVYYCARGDGWFDSWGQGTLVTVSS

SEQ ID NO: 26 - protein, 107 aa, synthetic construct
  REGION 1..107: note = Anti-HEWL Design 4 light chain variable
DIQLTQSPSFLSASVGDRVTITCRASQGISSNLAWYQQKPGKAPKLLIYYASTLQSGVPSRFSGSGSGTEFTLTISSLQPEDFATYYCQQSNSWPRTFGPGTKVDIK

SEQ ID NO: 27 - protein, 116 aa, synthetic construct
  REGION 1..116: note = Anti-HEWL Design 1,5,6 heavy chain variable
EVQLVQSGAEVKKPGATVKISCKVSGYTFSTYWMEWVQQAPGKGLEWMGEVLPGSGSTYYAEKFQGRVTITADTSTDTAYMELSSLRSEDTAVYYCARGDGYFQHWGQGTLVTVSS

SEQ ID NO: 28 - protein, 107 aa, synthetic construct
  REGION 1..107: note = Anti-HEWL Design 5 light chain variable
EIVMTQSPATLSVSPGERATLSCRASQSVSSNLAWYQQKPGQAPRLLIYYASTRATGIPARFSGSGSGTEFTLTISSLQSEDFAVYYCQQSNSWPRTFGPGTKVDIK

SEQ ID NO: 29 - protein, 107 aa, synthetic construct
  REGION 1..107: note = Anti-HEWL Design 6 light chain variable
EIVLTQSPDFQSVTPKEKVTITCRASQSISSNLHWYQQKPDQSPKLLIKYASQSFSGVPSRFSGSGSGTDFTLTINSLEAEDAATYYCHQSNSWPRTFGPGTKVDIK

SEQ ID NO: 30 - protein, 107 aa, synthetic construct
  REGION 1..107: note = Anti-PSA Clone 10 Light Chain variable
SIVMTQTPKILPVSAGDRVTMTCKASQSVVNNVVWYLQKPGQSPKLLIYYASNRYTGVPDRFTGSGSGTDFTFTISSVQVEDLTVYFCQQHYTSPLTFGAGTKLELK

SEQ ID NO: 31 - protein, 107 aa, synthetic construct
  REGION 1..107: note = Anti-PSA Design 1 Light Chain variable
DIQMTQSPSSLSASVGDRVTITCKASQSVVNNVVWYQQKPGKAPKLLIYYASNRYSGVPSRFSGSGSGTDFTLTISSLQPEDFATYYCQQHYTSPLTFGPGTKVDIK

SEQ ID NO: 32 - protein, 107 aa, synthetic construct
  REGION 1..107: note = Anti-PSA Design 2 Light Chain variable
DIVMTQTPLSLSVTPGQPASISCKASQSVVNNVVWYLQKPGQPPQLLIYYASNRYSGVPDRFSGSGSGTDFTLKISRVEAEDVGVYYCQQHYTSPLTFGPGTKVDIK

SEQ ID NO: 33 - protein, 107 aa, synthetic construct
  REGION 1..107: note = Anti-PSA Design 3 Light Chain variable
DIVMTQSPDSLAVSLGERATINCKASQSVVNNVVWYQQKPGQPPKLLIYYASNRYSGVPDRFSGSGSGTDFTLTISSLQAEDVAVYYCQQHYTSPLTFGPGTKVDIK

SEQ ID NO: 34 - protein, 107 aa, synthetic construct
  REGION 1..107: note = Anti-PSA Design 4 Light Chain variable
DIQMTQSPSSLSASVGDRVTITCKASQSVVNNVVWYQQKPGKAPKLLIYYASNRYSGVPSRFSGSGSGTEFTLTISSLQPEDFATYYCQQHYTSPLTFGPGTKVDIK

SEQ ID NO: 35 - protein, 107 aa, synthetic construct; identical to SEQ ID NO: 34
  REGION 1..107: note = Anti-PSA Design 5 Light Chain variable

SEQ ID NO: 36 - protein, 119 aa, synthetic construct
  REGION 1..119: note = Anti-PSA Clone 10 Heavy Chain variable
EVQLQESGPELVKPGASVMISCKTSGYSFTDYNMNWVKQNNGKSLDWIGVISPDYGTPSYNQKFKGKATLTVDQSSSTAYMELNSLTSEDSAVYYCARGKGNYVVFAYWGQGTLVTVAA

SEQ ID NO: 37 - protein, 119 aa, synthetic construct
  REGION 1..119: note = Anti-PSA Design 1 Heavy Chain variable
QVQLVQSGAEVKKPGSSVKVSCKASGYSFTDYNMNWVRQAPGQGLEWIGVISPDYGTPSYAQKFQGRVTITADESTSTAYMELSSLRSEDTAVYYCARGKGNYVVFAYWGQGTLVTVSS

SEQ ID NO: 38 - protein, 119 aa, synthetic construct; identical to SEQ ID NO: 37
  REGION 1..119: note = Anti-PSA Design 2 Heavy Chain variable

SEQ ID NO: 39 - protein, 119 aa, synthetic construct
  REGION 1..119: note = Anti-PSA Design 3 Heavy Chain variable
EVQLVQSGAEVKKPGATVKISCKVSGYSFTDYNMNWVQQAPGKGLEWIGVISPDYGTPSYAEKFQGRVTITADTSTDTAYMELSSLRSEDTAVYYCARGKGNYVVFAYWGQGTLVTVSS

SEQ ID NO: 40 - protein, 119 aa, synthetic construct
  REGION 1..119: note = Anti-PSA Design 4 Heavy Chain variable
QVQLQESGPGLVKPSETLSLTCTVSGYSFTDYNMNWIRQPPGKGLEWIGVISPDYGTPSYNPSLKSRVTISVDTSKNQFSLKLSSVTAADTAVYYCARGKGNYVVFAYWGQGTLVTVSS

SEQ ID NO: 41 - protein, 119 aa, synthetic construct; identical to SEQ ID NO: 39
  REGION 1..119: note = Anti-PSA Design 5 Heavy Chain variable

SEQ ID NO: 42 - protein, 118 aa, synthetic construct [sequence omitted]

SEQ ID NO: 43 - protein, 107 aa, synthetic construct
  REGION 1..107: note = 4IJ3 var. light, region Murine, not designed
DVVMTQSHKFMSTSVGDRVSITCKASQDVSTAVAWYQQKSGQSPKLLIHSASYRYTGVPDRFTGSGSGTDFTFTISSVQAEDLAVYYCQQHYSIPLTFGAGTKLELK

SEQ ID NO: 44 - protein, 467 aa, synthetic construct
  REGION 1..467: note = Ch3, heavy, Murine Fv, human Fc, Fv was designed
MGWSCIILFLVATATGVHCQVQLKQSGPGLVAPSQSLSITCTVSGFSLTGYGVNWVRQSPGKGLEWLGMIWGDGRTEYKSALKSRLSITKDNSKSQVFLKMNSLQTDDTARYYCASDYYGSGALEYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 45 - protein, 233 aa, synthetic construct
  REGION 1..233: note = Ch3, light, Murine Fv, human Fc, Fv was designed
MGWSCIILFLVATATGVHSDVVMTQSHKFMSTSVGDRVSITCKASQDVSTAVAWYQQKSGQAPKLLIHSASYRPTGVPDRFTGSGSGTDFTFTISSVQAEDLAVYYCQQHYSIPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 467 aa, synthetic construct
  REGION 1..467: note = Ch18, heavy, Murine Fv, human Fc, Fv was designed
MGWSCIILFLVATATGVHCQVQLKQSGPGLVAPSQSLSITCTVSGFSLTGYGVNWFRQSPGKGLEWLGMIWGDGRTDYKSALKSRLSITKDNSKSQVFLKMNSLQTDDTARYFCASDYYGSGSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 47 - protein, 233 aa, synthetic construct
  REGION 1..233: note = Ch18, light, Murine Fv, human Fc, Fv was designed
MGWSCIILFLVATATGVHSDVVMTQSHKFMSTSVGDRVSITCKASQDVSTAVAWYQQKSGQPPKLLIHSASYRVTGVPDRFTGSGSGTDFTFTISSVQAEDLAVYYCQQHYSIPFTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 467 aa, synthetic construct
  REGION 1..467: note = H2, Human Fc, humanized Fv
MGWSCIILFLVATATGVHCQITLKESGPTLVKPTQTLTLTCTFSGFSLTGYGVNWIRQPPGKALEWLGMIWGDGRTDYSPSLKSRLTITKDTSKNQVVLTMTNMDPVDTATYYCASDYYGSGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 49 - protein, 467 aa, synthetic construct
  REGION 1..467: note = H4, Human Fc, humanized Fv
MGWSCIILFLVATATGVHCQVQLQESGPGLVKPSETLSLTCTVSGFSLTGYGVNWIRQPPGKGLEWLGMIWGDGRTDYNPSLKSRVTISVDTSKNQFSLKLSSVTAADTAVYYCASDYYGSGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 50 - protein, 233 aa, synthetic construct
  REGION 1..233: note = K1a, Human Fc, humanized Fv
MGWSCIILFLVATATGVHSDIQMTQSPSSLSASVGDRVTITCKASQDVSTAVAWFQQKPGKAPKLLIHSASYRVTGVPSRFSGSGSGTDFTLTISSLQPEDFATYYCQQHYSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 233 aa, synthetic construct
  REGION 1..233: note = K1b, Human Fc, humanized Fv
MGWSCIILFLVATATGVHSAIQLTQSPSSLSASVGDRVTITCKASQDVSTAVAWYQQKPGKAPKLLIHSASYRVTGVPSRFSGSGSGTDFTLTISSLQPEDFATYYCQQHYSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 233 aa, synthetic construct
  REGION 1..233: note = K2, Human Fc, humanized Fv Human Fc, humanized Fv
MGWSCIILFLVATATGVHSDIVMTQTPLSLSVTPGQPASISCKASQDVSTAVAWYLQKPGQPPQLLIHSASYRVTGVPDRFSGSGSGTDFTLKISRVEAEDVGVYYCQQHYSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 233 aa, synthetic construct
  REGION 1..233: note = K3, Human Fc, humanized Fv
MGWSCIILFLVATATGVHSEIVMTQSPATLSLSPGERATLSCKASQDVSTAVAWYQQKPGQAPRLLIHSASYRVTGIPARFSGSGSGTDFTLTISSLQPEDFAVYYCQQHYSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 233 aa, synthetic construct
  REGION 1..233: note = K4, Human Fc, humanized Fv
MGWSCIILFLVATATGVHSDIVMTQSPDSLAVSLGERATINCKASQDVSTAVAWYQQKPGQPPKLLIHSASYRVTGVPDRFSGSGSGTDFTLTISSLQAEDVAVYYCQQHYSIPFTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 467 aa, synthetic construct
  REGION 1..467: note = H4 consensus, Human Fc, humanized Fv
MGWSCIILFLVATATGVHCQVQLQESGPGLVKPSETLSLTCTVSGFSLTGYGVNWIRQPPGKGLEWLGMIWGDGRTDYNPSLKSRVTISVDTSKNQFSLKLSSVTAADTAVYYCASDYYGSGSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 56 - protein, 233 aa, synthetic construct
  REGION 1..233: note = K1 consensus, Human Fc, humanized Fv
MGWSCIILFLVATATGVHSDIQMTQSPSSLSASVGDRVTITCKASQDVSTAVAWYQQKPGKAPKLLIHSASYRYSGVPSRFSGSGSGTDFTLTISSLQPEDFATYYCQQHYSIPLTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 467 aa, synthetic construct
  REGION 1..467: note = 492, heavy, Murine Fc, murine Fv
MGWSCIILFLVATATGVHCQVQLKQSGPGLVAPSQSLSITCTVSGFSLTGYGVNWVRQSPGKGLEWLGMIWGDGRTDYKSALKSRLSITKDNSKSQVFLKMNSLQTDDTARYFCASDYYGSGSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 58 - protein, 233 aa, synthetic construct
  REGION 1..233: note = 492, light, Murine Fc, murine Fv
MGWSCIILFLVATATGVHSDVVMTQSHKFMSTSVGDRVSITCKASQDVSTAVAWYQQKSGQSPKLLIHSASYRYTGVPDRFTGSGSGTDFTFTISSVQAEDLAVYYCQQHYSIPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 467 aa, synthetic construct; identical to SEQ ID NO: 1
  REGION 1..467: note = H2a, Human Fc, humanized Fv

SEQ ID NO: 60 - protein, 467 aa, synthetic construct; identical to SEQ ID NO: 2
  REGION 1..467: note = H2b, Human Fc, humanized Fv

SEQ ID NO: 61 - protein, 467 aa, synthetic construct; identical to SEQ ID NO: 3
  REGION 1..467: note = H3, Human Fc, humanized Fv

SEQ ID NO: 62 - protein, 234 aa, synthetic construct
  REGION 1..234: note = Anti-PSA Clone 9 Light chain, Human Fc, mouse Fv
MRVPAQLLGLLLLWLSGARCSIVMTQTPKILPVSAGDRVTMTCKASQSVVNNVVWYLQKPGQSPKLLIYYASNRYTGVPDRFTGSGSGTDFTFTISSVQVEDLTVYFCQQHYTSPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 234 aa, synthetic construct; identical to SEQ ID NO: 62
  REGION 1..234: note = Anti-PSA Clone 10 Light chain, Human Fc, mouse Fv

SEQ ID NO: 64 - protein, 468 aa, synthetic construct
  REGION 1..468: note = Anti-PSA Clone 10 Heavy chain, Human Fc, mouse Fv
MDWTWRFLFVVAAATGVQSEVQLQESGPELVKPGASVMISCKTSGYSFTDYNMNWVKQNNGKSLDWIGVISPDYGTPSYNQKFKGKATLTVDQSSSTAYMELNSLTSEDSAVYYCARGKGNYVVFAYWGQGTLVTVAAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 234 aa, synthetic construct
  REGION 1..234: note = Anti-PSA Clone 56 Light chain, Human Fc, mouse Fv
MRVPAQLLGLLLLWLSGARCSIVMTQTPKFLPVSAGDRVTMTCKASQSVVNNVVWYLQKPGQSPKLLIYYASNRYTGVPDRFTGSGSGTDFTFTISSVQVEDLAVYFCQQHYNSPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 469 aa, synthetic construct
  REGION 1..469: note = Anti-PSA Clone 56 Heavy chain, Human Fc, mouse Fv
MDWTWRFLFVVAAATGVQSEVQLQESGGGLVKPGGSLKLSCAASGFTFSTYAMSWVRQTPEKGLEWVAAISDSGTYTFYPDNVRGRFTISRDNAKNNLFLQMSHLKSEDTAMYYCTRDYYGSGSYWFFDVWGSGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 239 aa, synthetic construct
  REGION 1..239: note = Anti-PSA Clone 3 Light chain, Human Fc, mouse Fv
MRVPAQLLGLLLLWLSGARCDIVMTQAAFSNPVTLGTSASISCRSSKSLLHSDGITYLYWYLQRPGQSPQVLIYRMSNLASGVPDRFSGSGSGTDFTLRISRVEAEDVGVYYCAQMLEFPYTFGSGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 463 aa, synthetic construct
  REGION 1..463: note = Anti-PSA Clone 3 Heavy chain, Human Fc, mouse Fv
MDWTWRFLFVVAAATGVQSEVQLQESGPVLVKPGASVKMSCKASGYTFTDYYMSWVKQSHGKSLEWIGVVNPYSGGSTYNQKFKGKATLTVDKSSTTAYLELNSLTSEDSAVYYCARGAEDNWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 234 aa, synthetic construct; identical to SEQ ID NO: 11
  REGION 1..234: note = PSA1 Lc, Human Fc, humanized Fv

SEQ ID NO: 70 - protein, 468 aa, synthetic construct; identical to SEQ ID NO: 10
  REGION 1..468: note = PSA1 Hc, Human Fc, humanized Fv

SEQ ID NO: 71 - protein, 234 aa, synthetic construct; identical to SEQ ID NO: 12
  REGION 1..234: note = PSA2 Lc, Human Fc, humanized Fv

SEQ ID NO: 72 - protein, 468 aa, synthetic construct; identical to SEQ ID NO: 10
  REGION 1..468: note = PSA2 Hc, Human Fc, humanized Fv

SEQ ID NO: 73 - protein, 234 aa, synthetic construct; identical to SEQ ID NO: 14
  REGION 1..234: note = PSA3 Lc, Human Fc, humanized Fv

SEQ ID NO: 74 - protein, 468 aa, synthetic construct; identical to SEQ ID NO: 13
  REGION 1..468: note = PSA3 Hc, Human Fc, humanized Fv

SEQ ID NO: 75 - protein, 234 aa, synthetic construct; identical to SEQ ID NO: 16
  REGION 1..234: note = PSA4 Lc, Human Fc, humanized Fv

SEQ ID NO: 76 - protein, 468 aa, synthetic construct; identical to SEQ ID NO: 15
  REGION 1..468: note = PSA4 Hc, Human Fc, humanized Fv

SEQ ID NO: 77 - protein, 234 aa, synthetic construct; identical to SEQ ID NO: 16
  REGION 1..234: note = PSA5 Lc, Human Fc, humanized Fv

SEQ ID NO: 78 - protein, 468 aa, synthetic construct; identical to SEQ ID NO: 13
  REGION 1..468: note = PSA5 Hc, Human Fc, humanized Fv

SEQ ID NO: 79 - protein, 467 aa, synthetic construct; identical to SEQ ID NO: 57
  REGION 1..467: note = 492, heavy, Murine Fc, murine Fv

SEQ ID NO: 80 - protein, 233 aa, synthetic construct; identical to SEQ ID NO: 58
  REGION 1..233: note = 492, light, Murine Fc, murine Fv

SEQ ID NO: 81 - protein, 463 aa, synthetic construct; identical to SEQ ID NO: 68
  REGION 1..463: note = Anti-PSA Clone 3 Heavy chain, Human Fc, mouse Fv

SEQ ID NO: 82 - protein, 239 aa, synthetic construct; identical to SEQ ID NO: 67
  REGION 1..239: note = Anti-PSA Clone 3 Light chain, Human Fc, mouse Fv

SEQ ID NO: 83 - protein, 234 aa, synthetic construct; identical to SEQ ID NO: 62
  REGION 1..234: note = Anti-PSA Clone 9 Light chain, Human Fc, mouse Fv

SEQ ID NO: 84 - protein, 468 aa, synthetic construct
  REGION 1..468: note = Anti-PSA Clone 9 heavy chain, Human Fc, mouse Fv
MDWTWRFLFVVAAATGVQSEVQLQESGPELVKPGASVKISCTASGYSFTDYNMNWVKQNNGKSPEWIGVISPDYGTTSYNQNFKGRATLTVDQSSSTAYMQLNSLTSEDSAVYYCARGKGNYVVFAFWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 234 aa, synthetic construct; identical to SEQ ID NO: 62
  REGION 1..234: note = Anti-PSA Clone 10 Light chain, Human Fc, mouse Fv

SEQ ID NO: 86 - protein, 468 aa, synthetic construct; identical to SEQ ID NO: 64
  REGION 1..468: note = Anti-PSA Clone 10 Heavy chain, Human Fc, mouse Fv

SEQ ID NO: 87 - protein, 234 aa, synthetic construct; identical to SEQ ID NO: 65
  REGION 1..234: note = Anti-PSA Clone 56 Light chain, Human Fc, mouse Fv

SEQ ID NO: 88 - protein, 469 aa, synthetic construct; identical to SEQ ID NO: 66
  REGION 1..469: note = Anti-PSA Clone 56 Heavy chain, Human Fc, mouse Fv

SEQ ID NO: 89 - protein, 465 aa, synthetic construct
  REGION 1..465: note = Lysozyme Design 1 IgG1 heavy chain
MGWSCIILFLVATATGVHCEVQLVQSGAEVKKPGATVKISCKVSGYTFSTYWMEWVQQAPGKGLEWMGEVLPGSGSTYYAEKFQGRVTITADTSTDTAYMELSSLRSEDTAVYYCARGDGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 90 - protein, 233 aa, synthetic construct; identical to SEQ ID NO: 50
  REGION 1..233: note = K1a, Human Fc, humanized Fv

SEQ ID NO: 91 - protein, 467 aa, synthetic construct; identical to SEQ ID NO: 1
  REGION 1..467: note = H2a, Human Fc, humanized Fv

SEQ ID NO: 92 - protein, 467 aa, synthetic construct; identical to SEQ ID NO: 2
  REGION 1..467: note = H2b, Human Fc, humanized Fv

SEQ ID NO: 93 - protein, 467 aa, synthetic construct; identical to SEQ ID NO: 3
  REGION 1..467: note = H3, Human Fc, humanized Fv

SEQ ID NO: 94 - protein, 233 aa, synthetic construct; identical to SEQ ID NO: 5
  REGION 1..233: note = K1a, Human Fc, humanized Fv

SEQ ID NO: 95 - protein, 233 aa, synthetic construct; identical to SEQ ID NO: 6
  REGION 1..233: note = K2, Human Fc, humanized Fv

SEQ ID NO: 96 - protein, 233 aa, synthetic construct; identical to SEQ ID NO: 7
  REGION 1..233: note = K3, Human Fc, humanized Fv

SEQ ID NO: 97 - protein, 233 aa, synthetic construct; identical to SEQ ID NO: 8
  REGION 1..233: note = K4, Human Fc, humanized Fv

SEQ ID NO: 98 - protein, 233 aa, synthetic construct; identical to SEQ ID NO: 9
  REGION 1..233: note = K1b, Human Fc, humanized Fv

SEQ ID NO: 99 - protein, 233 aa, synthetic construct; identical to SEQ ID NO: 54
  REGION 1..233: note = K4, Human Fc, humanized Fv

SEQ ID NO: 100-101 - skipped (2 entries)

SEQ ID NO: 102 - protein, 107 aa, synthetic construct [sequence omitted]

SEQ ID NO: 103 - protein, 118 aa, synthetic construct
  REGION 1..118: note = D44.1 heavy variable
QVQLQESGAEVMKPGASVKISCKATGYTFSTYWIEWVKQRPGHGLEWIGEILPGSGSTYYNEKFKGKATFTADTSSNTAYMQLSSLTSEDSAVYYCARGDGNYGYWGQGTTLTVSSAS

SEQ ID NO: 104 - protein, 233 aa, synthetic construct
  REGION 1..233: note = D44.1 Mouse Fab, light
MGWSCIILFLVATATGVHSDIELTQSPATLSVTPGDSVSLSCRASQSISNNLHWYQQKSHESPRLLIKYVSQSSSGIPSRFSGSGSGTDFTLSINSVETEDFGMYFCQQSNSWPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 245 aa, synthetic construct
  REGION 1..245: note = D44.1 Mouse Fab, heavy
MGWSCIILFLVATATGVHCQVQLQESGAEVMKPGASVKISCKATGYTFSTYWIEWVKQRPGHGLEWIGEILPGSGSTYYNEKFKGKATFTADTSSNTAYMQLSSLTSEDSAVYYCARGDGNYGYWGQGTTLTVSSASTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGKHHHHHH

SEQ ID NO: 106 - protein, 465 aa, synthetic construct; identical to SEQ ID NO: 89
  REGION 1..465: note = Anti-HEWL Design 6 IgG1, Human Fc, humanized Fv, heavy

SEQ ID NO: 107 - protein, 233 aa, synthetic construct
  REGION 1..233: note = Anti-HEWL Design 6 IgG1, Human Fc, humanized Fv, light
MGWSCIILFLVATATGVHSEIVLTQSPDFQSVTPKEKVTITCRASQSISSNLHWYQQKPDQSPKLLIKYASQSFSGVPSRFSGSGSGTDFTLTINSLEAEDAATYYCHQSNSWPRTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0122311 A1
(43) Pub. Date: Apr. 17, 2025

(54) METHOD FOR HUMANIZING ANTIBODIES

(71) Applicant: YEDA RESEARCH AND DEVELOPMENT CO. LTD., Rehovot (IL)

(72) Inventors: Sarel FLEISHMAN, Rehovot (IL); Ariel TENNENHOUSE, Rehovot (IL)

(21) Appl. No.: 18/681,143

(22) PCT Filed: Aug. 5, 2022
(86) PCT No.: PCT/IL2022/050854

§ 371 Date: Feb. 5, 2024

Related U.S. Application Data
(60) Provisional application No. 63/230,089, filed on Aug. 6, 2021.

(51) Int. Cl. C07K 16/46; C07K 16/30; C07K 16/40

(52) U.S. Cl. CPC C07K 16/464; C07K 16/3069; C07K 16/40; C07K 16/462; C07K 2317/24; C07K 2317/565; C07K 2317/92; C07K 2317/94

(57) ABSTRACT

Provided herein is a method for obtaining a humanized antibody based on a non-human antibody having an affinity to an antigen of interest, that starts from an experimental or model structure, grafts the non-human CDRs on a library of human frameworks and uses restrained/constrained atomistic simulations to relax and rank the designs by energy.

### RELATED APPLICATION

This application claims the benefit of priority of U.S. Provisional Patent Application No. 63/230,089 filed Aug. 6, 2021, the contents of which are incorporated herein by reference in their entirety.

### SEQUENCE LISTING STATEMENT

The ASCII file, entitled txt_93036.txt, created on Aug. 4, 2022, comprising 162,676 bytes, submitted concurrently with the filing of this application is incorporated herein by reference.

### FIELD AND BACKGROUND OF THE INVENTION

The present invention, in some embodiments thereof, relates to a computational method for designing antibodies, and more particularly, but not exclusively, to a method for humanizing antibodies.

Antibodies are the largest segment of protein-based therapeutics with over 100 in clinical use or under regulatory review. As many as 40% of these antibodies were isolated from an animal source, mostly murine/mouse) and were humanized prior to clinical application. Antibody humanization is essential to achieve three important therapeutic goals: recruiting the immune system through Fc effector functionality; increasing blood circulation half-life; and mitigating immunogenicity in cases in which the antibody is destined for long-term treatments.

Despite the critical role of antibody humanization, it is an iterative and often frustrating process. The first step chimerizes the animal variable domain (Fv) with human constant domains. Next, the Fv, which comprises more than 200 amino acids, is humanized. This step is often complicated by the fact that the Fv comprises the so-called complementarity-determining regions (CDRs) which are responsible for antigen recognition. Therefore, the mainstream humanization strategy grafts the CDRs from the animal source on a human framework, typically leading to an Fv with more than 80% sequence identity to the human germline (compared to 50-70% identity for a mouse Fv). To increase the chances that the grafted CDRs are compatible with the human framework, the latter are typically picked from those showing the highest homology to the parental antibody. Other approaches to antibody humanization use structure similarity in the CDR regions rather than sequence homology, humanize only predicted immunogenic segments in the parental framework, or graft fragments from human frameworks into the animal antibody.

Despite these advances, however, Fv humanization typically leads to substantial, sometimes orders of magnitude decrease in expression levels, stability and affinity. The deterioration in the antibody's biophysical properties is especially detrimental in the context of an antibody that is destined for clinical use as it leads to reduction in efficacy, and can lead to undesirable complications in formulating and delivering the drug. As a rule, therefore, a third step of “backmutation” mutates positions in the humanized antibody to their parental identities through iterative design-and-experiment cycles.

The underlying reason for the loss in affinity and stability through humanization is structural and energetic. Structural analyses singled out the importance of a region of the framework called the vernier zone, which underlies the CDRs. Despite the relatively high conservation of the framework, the vernier zone comprises approximately 30 sequence determinants that vary even among homologous frameworks; these determinants are essential for the structural integrity and relaxation of the CDRs. Thus, most backmutation attempts use structural modeling to select mutations that reconstitute some of the vernier-zone positions seen in the animal antibody. This process can regain the parental antibody's affinity and stability, though at the cost of lower humanness and lengthy iterations.

U.S. Pat. No. 8,343,489 describes the use of three-dimensional structure information to guide the process of modifying antibodies with amino acids from one or more templates or surrogates such that the antigen binding properties of the parent antibody are maintained and the immunogenicity potential is reduced when administered as a therapeutic in humans.

International Patent Application No. WO 2019/025299 provides a method for the humanization of non-human antibodies using a structure-based scoring matrix, with which it is possible to determine the requirement for and the suitability of specific back-mutations of amino acid residues at defined positions of a selected human germline sequence. The scoring matrix takes into account the topology, the three-dimensional structure and the interactions of the respective residue and change; thereby the influence on antigen binding of a specific amino acid residue change can be determined.

### SUMMARY OF THE INVENTION

Humanization is an essential step in developing animal-derived antibodies into therapeutics, and approximately 40% of FDA-approved antibodies have been humanized. Conventional humanization approaches graft the complementarity-determining regions (CDRs) of the animal antibody onto a few dozen homologous human frameworks. This process, however, often drastically lowers stability and antigen binding, demanding iterative mutational fine-tuning to recover the original antibody's properties. The method presented herein is a computational hUMan AntiBody design (“CUMAb”), is a method that starts from an experimental or model antibody structure, grafts the animal CDRs on thousands of human frameworks and uses Rosetta atomistic simulations to rank the designs by energy and structural integrity.

The present disclosure thus provides a method for designing antibodies to be compatible for use in humans albeit they originate in another species, namely a method for humanizing antibodies, which is based on structural and energy based ranking rather than on the commonly used sequence homology. Starting from experimentally determined or computed model structure of a non-human Ab, and a database of human antibody germline sequences, the method includes generating a large number of grafted structures and then uses atomic structure design calculations, such as provided in the Rosetta package, to relax, score and rank the humanized grafted designed variants based on their energetic stability score. Crucially, automation allows the method to expand humanization from a few dozen homologous frameworks to as many as 20,000 different ones. Proof-of-concept (POC) experiments showed that some of the top-ranked designs for three unrelated targets (for which classical humanization methods have failed) exhibited expression levels and binding properties on par with the parental animal antibodies without involving iterative backmutation processes. In all cases used for POC, the experimentally best-performing humanized design has been derived from a human framework that is not necessarily of the highest homology with respect to the parent Ab, suggesting that energy-based humanization may solve problems seen in conventional, homology-based humanization. The herein provided method, is also called Computational hUMan AntiBody design (CUMAB).

Thus, according to an aspect of some embodiments of the present invention, there is provided a method for designing and producing a humanized antibody having an affinity to an antigen of interest, which is effected by:
i) providing a structural model of a non-human antibody having an affinity to the antigen of interest (a parental Ab) and identifying amino acid residues of at least one complementarity-determining region (CDR) in the structural model;
ii) generating all combinations of antibody segments derived from a plurality of human antibody germline sequences, and replacing corresponding amino acid residues in each of the combinations with the amino acid residues of the CDR, to thereby obtain a library of grafted human antibody sequences;
iii) threading each of the grafted human antibody sequences on the structural model to thereby obtain a plurality of threaded grafted human antibody structures, and subjecting each of the threaded grafted human antibody structures to constrained energy minimization (constrained structural relaxation) to thereby obtain a plurality of relaxed grafted human antibody structures;
iv) ranking the plurality of relaxed grafted human antibody structures by an energy score;
v) clustering the plurality of relaxed grafted human antibody structures according to V/J gene families to thereby obtain an energy ranked and gene family clustered library of humanized antibody designs; and
vi) expressing at least one humanized antibody design from at least one cluster of humanized antibody designs, and selecting at least one humanized antibody design having an affinity to the antigen of interest, thereby obtaining the humanized antibody having an affinity to the antigen of interest.

In some embodiments, the method further includes, prior to the threading, step, subjecting the structural model to energy minimization (constrained structural relaxation).

In some embodiments, the antibody segments are selected from the group consisting of heavy chain variable (V) gene segment, light chain variable (V) gene segment, heavy chain joining (J) gene segment, light chain joining (J) gene segment, kappa gene segment, and lambda gene segment.

In some embodiments, the method further includes removing (filtering-out) sequences that exhibit more than two cysteines outside the CDR from the library of grafted human antibody sequences.

In some embodiments, the method further includes removing (filtering-out) sequences that exhibit Asn-Gly or Asn-X-Ser/Thr (where X is not Pro) motifs from the library of grafted human antibody sequences.

In some embodiments, the method further includes removing (filtering-out) from the plurality of relaxed grafted human antibody structures a structure exhibiting more than 0.5 Å RMSD in a backbone atom of the CDR compared to the structural model of a non-human antibody.

In some embodiments, the plurality of human antibody germline sequences is obtainable from a human genetics database.

In some embodiments, the human genetics database is the immunogenetics and immunoinformatics IMGT database.

In some embodiments, the non-human antibody is a mouse antibody.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings and images in detail, it is stressed that the particulars shown are by way of example and for purposes of illustrative discussion of embodiments of the invention. In this regard, the description taken with the drawings and images makes apparent to those skilled in the art how embodiments of the invention may be practiced.

In the drawings:

FIGS. 1A-C present structural aspects of CDR grafting, wherein FIG. 1A presents a cartoon depicting of the domains of an antibody and the inset shows a crystal structure of an antibody variable region (4IJ3; SEQ ID Nos (heavy/light): 43/42) with heavy chain colored blue and light chain colored pink and CDRs are highlighted in bright blue and bright pink according to their chain, whereas the schematic below the inset shows the breakdown of V and J genes within each chain, and FIG. 1B presents a table comparing CUMAB CDR definitions to those from Kabat using Kabat numbering, and FIG. 1C presets the distribution of energies of sequences produced in a typical CDR grafting run-shown in lines are the energies of the parental sequence after the same protocol, the top 5 clustered designs, and the remaining combinations of the light and heavy chain sequences that are found in the top 5 designs;

FIGS. 2A-C present the first attempt of using the presently provided method for Ab humanization on an anti-QSOX1 antibody: FIG. 2A. Dot blot of 10 designs (SEQ ID Nos (heavy chain/light chain): 48/50, 48/51, 48/52, 48/53, 48/54, 49/50, 49/51, 49/52, 49/53, 49/54, designs label starting with “K”, some are shown in duplicates) as well as chimera 3 and 18 from the AbLift stabilization of the chimeric antibody, and some failed designs from an earlier approach (starting with “L”), expressed as full length IgG1s in HEK293 cells; FIG. 2B. The designs and the two chimeras were tested for QSOX1 inhibition by incubating QSOX1, the antibody, and a 55 kDa substrate with two cysteines. The reaction was stopped by addition of PEG-maleimide 5k, which adds ˜30 kDa of apparent mass to the protein per pair of reduced cysteines. When QSOX1 is present and active, the cysteines are oxidized and there is only the band at 55 kDa. When QSOX1 is absent or inhibited, the cysteines are reduced and there is also a band at ˜85 kDa. FIG. 2C. Electrophoretic analysis of the 10 designs and the two chimeras in denaturing conditions. Bands are expected to be present at ˜25 kDa (light chain) and ˜50 kDa (heavy chain);

FIGS. 3A-F present anti-QSOX1 antibody humanization: FIG. 3A. 15 humanized designs were expressed and expression levels measured by dot blot (Table 4; SEQ ID Nos. (heavy chain/light chain): 1/5, 1/6; 1/7, 1/8, 1/9, 2/5, 2/6, 2/7, 2/8, 2/9, and 3/5, 3/6, 3/7, 3/8, 3/9). Chim 18 (SEQ ID NO: 46/47) is a chimeric version of the antibody with additional stabilizing mutations that was previously shown to have identical expression levels to the parental antibody (SEQ ID NO: 57/58). FIG. 3B. The designs were run on a non-denaturing gel. The expected size of each of the antibodies is approximately 150 kDa. 23. FIG. 3C. The designs were screened for QSOX1 inhibition by incubating the antibody, QSOX1, and the QSOX1 substrate ZG16. The reaction was stopped by the addition of PEG-maleimide 5k, which adds approximately 30 kDa of apparent mass if the cysteines are reduced. QSOX1 activity is seen in slower electrophoretic mobility (higher band) of ZG16, and conversely, its inhibition manifests in faster mobility (lower band). FIG. 3D. Four best designs from the prior screen were expressed in larger cultures, purified, and run on non-denaturing gel (SEQ ID Nos. (heavy chain/light chain): 1/6, 2/8, 3/6, 1/6 (repetition), and 3/8). FIG. 3E. QSOX1 inhibition was measured by incubating antibodies (SEQ ID Nos (heavy chain/light chain): 79/80, 2/8 and 3/8; various concentrations) and QSOX1 (25 nM). Reaction activated by adding DTT and then stopped by adding DTNB. Absorbance at 412 nm was measured and reflects the number of free thiols from DTT that reacted with DTNB. Plotted is inhibition fraction=Absorbance/(Absorbance no QSOX added-Absorbance no antibody added) FIG. 3F. Dot-blot analysis of anti-QSOX1 CDRs grafted onto most homologous consensus frameworks IGKV1-IGHV4 (Seq NO: 56/55) compared to a mock transfection and well-expressed AbLIFT design 3 (Seq NO: 44/45);

FIGS. 4A-E present anti-PSA clone 10 humanization: FIG. 4A. Four anti-PSA antibodies were expressed as chimeras and expression was measured through ELISA of the supernatant SEQ ID Nos.: 83/84, 85/86, 87/88, 81/82). Clone 3 (heavy and light chains denoted SEQ ID Nos. 68 and 67, respectively) showed strong expression while clones 9, 10, and 56 (heavy and light chains denoted SEQ ID Nos. 84/83, 86/85, and 88/87 respectively) showed no expression. FIG. 4B. Expression of five humanized designs for clone 10 was measured through ELISA of the supernatant. All five designs showed high expression levels (PSA1-PSA5 having heavy and light chain SEQ ID Nos.: 70/69, 72/72, 74/73, 76/75, and 78/77, respectively). FIG. 4C. Binding of the five humanized designs (PSA1-PSA-5) for clone 10 (after purification with protein G) to recombinant PSA was measured using ELISA. FIG. 4D. Immunoprecipitation of five humanized designs for clone 10 (PSA1-PSA5 having heavy and light chain SEQ ID Nos. (heavy/light): 70/69, 72/72, 74/73, 76/75, and 78/77, respectively) with protein G after incubation with recombinant PSA. Membrane was developed with a commercial anti-PSA antibody. Recombinant PSA is known to be ˜35 kDa and there is a degradant of the protein about 25 kDa. FIG. 4E. Antibodies (Ayelet79 and PSA4-SEQ ID Nos.: 76/75) were incubated with PSA overnight (Rec or native in LNCaP supernatant) and immunoprecipitated with protein G. Membrane was developed with a commercial anti-PSA antibody. The native PSA is ˜35 kDa with an additional higher band for pre-PSA;

FIGS. 5A-D present anti lysozyme antibody SDR-grafting: FIG. 5A. Histogram of Binding from Yeast display screen of 6 designs (SEQ ID Nos (heavy chain/light chain): 27/22, 21/22, 23/24, 25/26, 27/28, 27/29) as well as a negative control (G6) and positive control (D44.1, SEQ ID Nos 103/102) with lysozyme concentration of 240 nM. FIG. 5B. Denaturing gel of D44.1 and Des6 (SEQ ID NO: 106/107) expressed as full length IgG1s after purification with protein G. 3 elutions were done for each sample. Bands are expected to be present at ˜25 kDa (light chain) and ˜50 kDa (heavy chain). FIG. 5C. Denaturing gel of Des1 (SEQ ID NO: 19/20) expressed as full length IgG1 after purification with protein G. 4 elutions were done and are shown on the gel from left to right. Bands are expected to be present at ˜25 kDa (light chain) and ˜50 kDa (heavy chain). FIG. 5D. Bilayer interferometry traces for Des 1 (SEQ ID NO: 19/20). Lysozyme was at concentrations of 1000, 250, 100, 25, and 10 nm and 1 μg/mL antibody was loaded onto the sensor. Kd calculated to be 11 nM with error of 0.31 nM;

FIG. 6 presents a schematic flowchart of the method provided herein, according to some embodiments of the present invention; and

FIG. 7 presents a comparative bar-graph, comparing the activity (affinity) assay results conducted for the expressed antibodies 492 (SEQ ID Nos. (heavy/light): 79/80), chimera, h2bK4 (SEQ ID Nos.: 2/8), h3K4 (SEQ ID Nos.: 3/8), h3newK2 (SEQ ID Nos.: 4/6) and h3newK4 (SEQ ID Nos.: 4/8).

### DESCRIPTION OF EMBODIMENTS OF THE INVENTION

The present invention, in some embodiments thereof, relates to a computational method for designing antibodies, and more particularly, but not exclusively, to a method for humanizing antibodies.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

As discussed hereinabove, antibody humanization is critical for developing animal-derived antibodies into therapeutics, and 40% of clinically approved antibodies were humanized. Conventional approaches for humanization graft the complementarity-determining regions (CDRs) of the animal antibody onto a few dozen homologous human frameworks. Despite this approach's success and importance, grafting often substantially decreases stability, expression levels and binding affinity or specificity, demanding iterative mutational fine-tuning to recapitulate the parental antibody's properties.

The present disclosure provides a computational method that uses as input an experimental or model structure, grafts the animal CDRs on thousands of human frameworks and uses, for example, Rosetta atomistic simulations to relax and rank the designs by energy. The reduction to practice of the herein-provided method afforded designs that exhibit identical affinity to the animal antibody, even in cases where conventional antibody humanization failed to produce expressible antibodies, let alone high-affinity binders. The successful design of dozens of mutations in the antibody variable domain suggests that other critical antibody engineering methods may be amenable to design automation. The method can be made accessible to the public via a webserver to help rationalize antibody engineering pipelines.

Unlike other computational methods for humanization of antibodies (Abs) which require an experimentally obtained structure of the parental (source) antibody (Ab), the method presented herein (also referred to as “CUMAb”) does not rely on such starting point. Computational methods for Ab humanization, such as the one presented in U.S. Pat. No. 8,343,489, are effected by aligning the structure of the parental antibody to the antibody structures in the Protein Data Bank to find a closest match, and further in structure grafting method of the entire antibody and in the EPU method for each of the CDRs and the framework separately. The method presented herein, according to some embodiments of the present invention, does not rely on structures of other antibodies; this is advantageous since relying on structures of other antibodies can be severely limiting and misleading.

Computational methods for Ab humanization, such as the one presented in U.S. Pat. No. 8,343,489 and WO 2019/025299, rely on sequence homology to choose from the structures selected previously, and the CDR definitions may also bias the end result. Furthermore, such methods require many intense visual inspection steps of the antibodies, rendering such method unsuitable for high throughput endeavors.

The method provided herein (CUMAb) is based on the fundamental insight that antibody stability and activity are determined both by the CDRs and by the amino acid positions on which the CDRs rest. This insight may help address other important challenges in antibody engineering leading to general, reliable and automated antibody design strategies.

CUMAb designs of several independent antibodies exhibit similar affinity to the animal antibody even where conventional antibody humanization failed to produce an expressible antibody. Low-energy but nonhomologous frameworks are often preferred to the highest-homology ones, and several CUMAb designs encoding dozens of mutations from one another are functionally equivalent. Surprisingly, some designs show marked improvement in stability and expressibility relative to the parental antibodies. Thus, CUMAb presents a general and streamlined approach to optimize antibody stability and expressibility while increasing humanness.

Instead of using only a few dozen homologous frameworks as in the majority of conventional humanization strategies, the method presented herein (CUMAb) uses all combinations of possible human gene segments (>20,000 for each antibody) and ranks them by energy. Although genes belonging to a single subgroup are similar to one another, they contain mutations, including in vernier positions that may stabilize the specific CDRs of the parental antibody. Structural analysis shows that low-energy designs retain critical framework-CDR interactions that may be eliminated in homology-based humanization. Consequently, the lowest-energy designs from this large space of possible frameworks are more likely than the highest homology ones to retain stability, expression yields, and binding affinity, and in even improve stability and expressibility. CUMAb may thus offer a strategy to improve antibody stability, including of human-sourced antibodies, while maintaining or increasing humanness.

Crucially, the energy-based strategy eliminates iterative back mutation even when starting from an Fv model structure. Thus, CUMAb increases the scope of antibody humanization, in principle, to the sequence of any animal antibody. Furthermore, CUMAb is automated and requires experimental screening of fewer than a dozen constructs substantially reducing time and cost. It may therefore be applied at scale to dozens of antibodies in parallel, including ones for which a structure is not available. We also note that in the three cases we examined, several unique humanization alternatives exhibited stability and binding properties that were on a par with those of the animal antibody. These designs provide alternatives for selecting the best-behaved humanized antibody variant in downstream experiments because they are different from one another by dozens of surface mutations.

The method provided herein (CUMAb) relies on readily available input data, it can be set for full automation, and it can easily be scaled to many antibodies and be used by non-experts.

### Energy-Based Ranking of Humanized Antibodies:

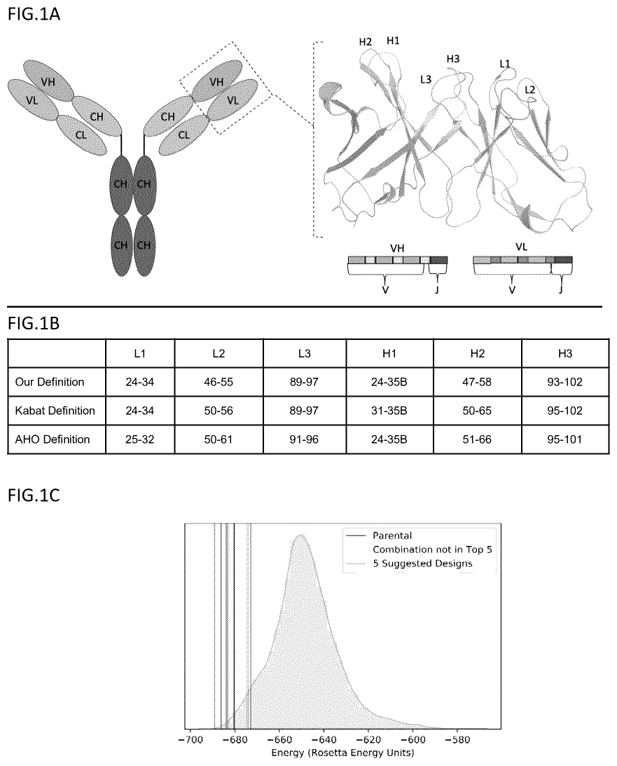

A computational workflow was developed for modeling and energy-ranking structures in which the Fv framework regions are replaced with all compatible human frameworks. The framework is encoded in two gene segments, V and J, on both the light and heavy chains (FIG. 1A) [Janeway, C. A.; Travers, P.; Walport, M.; Shlomchik, M. J. Immunobiology: The Immune System in Health and Disease, 6th edition; Garland Science: New York, 2005]. Recombination of all the human V and J segments on both light and heavy chains gives rise to tens of thousands of unique frameworks (63,180 that comprise kappa light chains and 48,600 for lambda light chains) [Lefranc, M. P. Nomenclature of the Human Immunoglobulin Kappa (IGK) Genes. Exp. Clin. Immunogenet. 2001, 18 (3), 161-174]. The working hypothesis of this study is that the energy-based ranking of so many different human frameworks may yield humanized designs that are more stable and functional than those from conventional, homology-based humanization.

CDRs were defined based on combinations of definitions provided elsewhere [MacCallum, R. M.; Martin, A. C.; Thornton, J. M. Antibody-Antigen Interactions: Contact Analysis and Binding Site Topography. J. Mol. Biol. 1996, 262 (5), 732-745; Dondelinger, M.; Filée, P.; Sauvage, E.; Quinting, B.; Muyldermans, S.; Galleni, M.; Vandevenne, M. S. Understanding the Significance and Implications of Antibody Numbering and Antigen-Binding Surface/Residue Definition. Front. Immunol. 2018, 9, 2278] (FIG. 1A-B).

The use of the method provided herein starts by exchanging the amino acid sequences in regions outside the CDRs with all combinations of the human V and J sequences obtained from the ImMunoGeneTics (IMGT) database. The light chains are humanized using either lambda or kappa light chains according to the light-chain class of the animal antibody. Genes that contain Asn-Gly or Asn-X-Ser/Thr (where X is not Pro) sequence motifs were eliminated as these may lead to undesirable post-translational modifications. Additionally, any sequence that exhibits more than two cysteines outside of the CDRs is excluded to reduce the chances of antibody misfolding or aggregation. These restrictions retain a large fraction of possible combinations of human genes resulting in >20,000 unique frameworks per antibody.

For the grafting step, the human and animal V and J genes were aligned and the parental (non-human, animal) CDR amino acids replaced their human counterparts. The result is library of grafted human antibody sequences.

Starting from the structure of the parental (non-human) antibody Fv, each humanized design is modeled using Rosetta all-atom calculations by threading the sequence of the humanized design onto the structure of the parental antibody [Leaver-Fay, A.; Tyka, M.; Lewis, S. M.; Lange, O. F.; Thompson, J.; Jacak, R.; Kaufman, K.; Renfrew, P. D.; Smith, C. A.; Sheffler, W.; Davis, I. W.; Cooper, S.; Treuille, A.; Mandell, D. J.; Richter, F.; Ban, Y.-E. A.; Fleishman, S. J.; Corn, J. E.; Bradley, P. ROSETTA3: An Object-Oriented Software Suite for the Simulation and Design of Macromolecules. Methods Enzymol. 2011, 487, 545-574]. The result of this step is a plurality of threaded grafted human antibody structures.

The resulting model structure is relaxed through cycles of sidechain and harmonically constrained backbone minimization and combinatorial sidechain packing in the entire Fv. Each model is ranked using the ref2015 energy function [O'Meara, M. J.; Leaver-Fay, A.; Tyka, M. D. M.; Stein, A.; Houlihan, K.; DiMaio, F.; Bradley, P.; Kortemme, T.; Baker, D.; Snoeyink, J.; Kuhlman, B. A Combined Covalent-Electrostatic Model of Hydrogen Bonding Improves Structure Prediction with Rosetta. J. Chem. Theory Comput. 2015, 11 (2), 609-622] which is dominated by van der Waals interactions, hydrogen bonding, electrostatics, and implicit solvation. The result of this step is a plurality of relaxed grafted human antibody structures.

Models in which any of the CDR backbone conformations deviates by more than 0.5 Å from the parental conformation are eliminated. To select a diverse set of sequences for experimental testing, the top-ranked designs were clustered according to V-gene subgroups which are defined according to sequence homology (7, 6 and 10 clusters for heavy V, kappa and lambda). This clustering yields a short list of diverse, low-energy models for experimental testing, also referred to herein as an energy ranked and gene family clustered library of humanized antibody designs.

Of note, unlike conventional CDR-grafting methods, the approach taken by the method provided herein is agnostic to homology between the mouse and human framework and is scalable, designing and ranking 20 thousand different humanized constructs on a 500-CPU cluster within a few hours.

The next step may be expressing at least one humanized antibody design from at least one cluster of the resulting humanized antibody designs, and selecting at least one humanized antibody design. The criteria for a successful use of the method provided herein may include an expression level assay, inferring on the stability of the Ab protein, and assaying the affinity of the expressed Ab to the antigen of interest, and comparing this affinity to that of the parental Ab.

### CDR Grafting Based on a Crystal Structure:

As an exemplary humanization target, an antibody that was raised through mouse immunization to target human Quiescin Sulfhydryl Oxidase 1 (QSOX1) was chosen [Grossman, I.; Alon, A.; Ilani, T.; Fass, D. An Inhibitory Antibody Blocks the First Step in the Dithiol/disulfide Relay Mechanism of the Enzyme QSOX1. J. Mol. Biol. 2013, 425 (22), 4366-4378]. This antibody is challenging for humanization since chimerizing its mouse Fv with a human IgG1 constant region leads to a complete loss of expression in HEK293 cells. This challenge has been addressed previously with the AbLIFT method, which uses atomistic design calculations to improve the molecular interactions between the Fv light and heavy domains [Warszawski, S.; Borenstein Katz, A.; Lipsh, R.; Khmelnitsky, L.; Ben Nissan, G.; Javitt, G.; Dym, O.; Unger, T.; Knop, O.; Albeck, S.; Diskin, R.; Fass, D.; Sharon, M.; Fleishman, S. J.: Optimizing Antibody Affinity and Stability by the Automated Design of the Variable Light-Heavy Chain Interfaces. PLOS Comput. Biol. 2020, 16 (10), e1008382]. One of the designs, AbLIFT18, rescued HEK293 expression and QSOX1 inhibition but presented an incompletely humanized antibody.

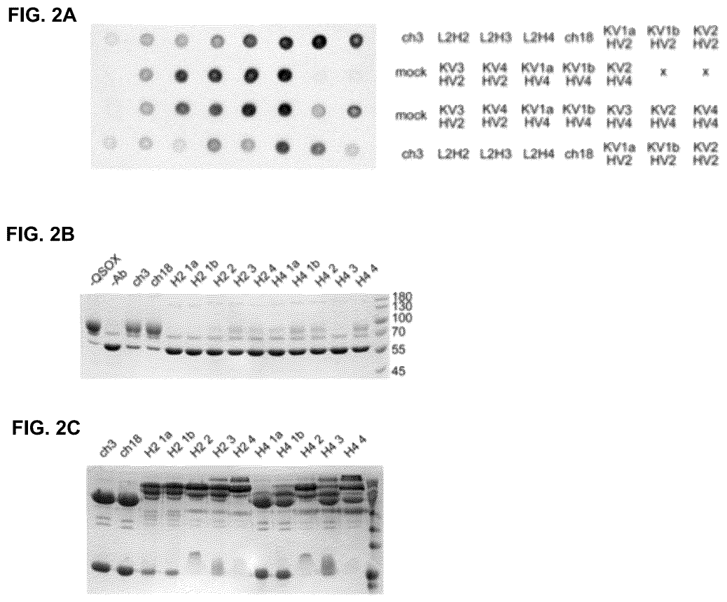

Using a preliminary version of the humanization workflow provided herein, ten designs were tested experimentally in the following combinations: SEQ ID Nos. (heavy chain/light chain): 48/50, 48/51, 48/52, 48/53, 48/54, 49/50, 49/51, 49/52, 49/53, and 49/54, comprising the lowest-energy five designs after clustering as well as all of the combinations of heavy and light chains observed in these five; these combinations were all within the top 2,700 of over 26,000 designs (ranking using updated method). The difference between this preliminary protocol and the current was that the H1 CDR definition in the preliminary version excluded two N-terminal amino acid positions relative to the final protocol. In addition, in this exemplary use of the method provided herein, CDR mutations that were implemented in the AbLIFT18 design were incorporated relative to the mouse antibody. These ten designs (SEQ ID Nos. (heavy chain/light chain): 48/50, 48/51, 48/52, 48/53, 48/54, 49/50, 49/51, 49/52, 49/53, and 49/54) were formatted as IgG1 full-length antibodies and expressed in HEK293 cells, followed by protein G affinity purification. A qualitative dot-blot analysis showed that many designs expressed as well as AbLIFT18 but none showed comparable QSOX1 inhibition levels (FIGS. 2A and 2B). Electrophoretic-mobility analysis in denaturing conditions revealed that the designs' apparent molecular mass was heterogeneous and different from that of the parental antibody, suggesting that these designs were aggregated or misfolded (FIG. 2C). Visual inspection of the design models to find the source of these stability problems revealed that one of the humanization mutations, heavy chain Val24Phe (Kabat numbering), which was present in nearly all of the designs, was structurally incompatible with the conformation of CDR H1 in the parental antibody, suggesting that the preliminary CDR definitions failed to include amino acids that were important for humanization.

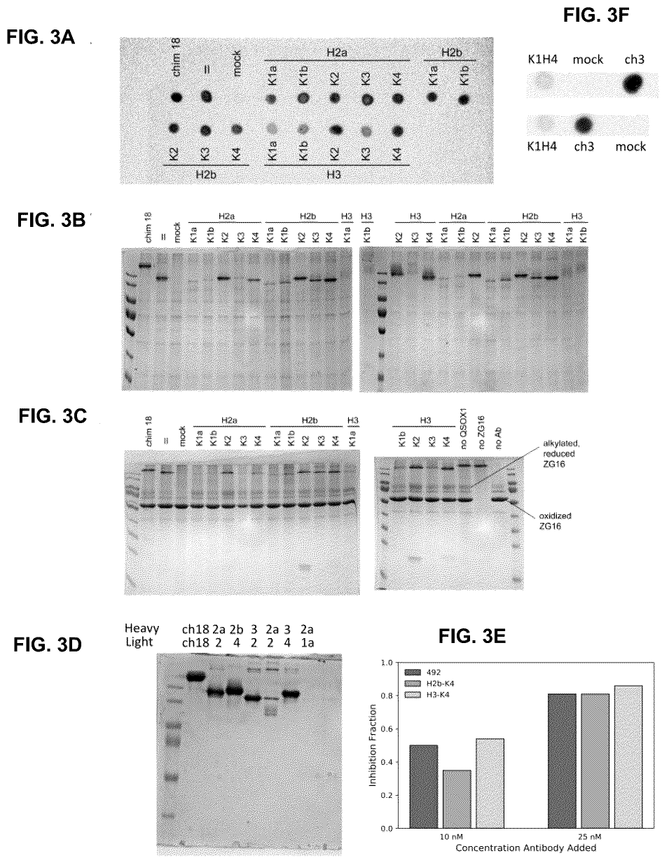

The design calculations were repeated using the CUMAB final version of the CDR definitions which incorporated Kabat positions 24 and 25 in the H1 definition (FIG. 1A-B). This method run started directly from the parental mouse antibody without including the mutations in the CDRs from design AbLIFT18. Fifteen designs were experimentally tested (top-five ranked designs and the combinations of light and heavy chains from these five; Table 4) of which 12 showed comparable expression levels on a dot-blot analysis to design AbLIFT18 (FIG. 3A; SEQ ID Nos. (heavy/light): 1/5, 1/6, 1/7, 1/8, 1/9, 2/5, 2/6, 2/7, 2/8, 2/9, 3/6, and 3/8).

Unlike the first humanization attempt, electrophoretic analysis revealed that seven designs showed comparable expression levels to AbLIFT18 with no significant evidence of misfolding or aggregation (FIG. 3B; Nos. (heavy/light): 1/6, 1/8, 2/6, 2/7, 2/8, 3/6, 3/8). Remarkably, two of the designed antibodies denoted H2B-K4 and H3-K4 (SEQ ID Nos. (heavy/light): 2/8 and 3/8, respectively) showed equivalent levels of QSOX1 inhibition to the parental mouse antibody (492, SEQ ID Nos. (heavy/light): 79/80) when added at stoichiometric ratios and one exhibited substantial levels of inhibition even at sub stoichiometric ratios (FIG. 3E). Thus, these two designs recapitulated the parental mouse expression and activity using a humanized Fv framework. These results also emphasize the importance of structurally appropriate CDR definitions for humanization success.

### Humanization Based on an Fv Model Structure:

Experimental structure determination is a lengthy and uncertain process. In many antibody-discovery studies, obtaining an experimentally determined structure for each antibody is unrealistic. Recently, structure-prediction methods have reached the point in which the antibody Fv structure can be predicted to nearly atomic accuracy except in the solvent-exposed parts of CDR H3 where models are still insufficiently reliable [Almagro, J. C.; Teplyakov, A.; Luo, J.; Sweet, R. W.; Kodangattil, S.; Hernandez-Guzman, F.; Gilliland, G. L. Second Antibody Modeling Assessment (AMA-II). Proteins: Struct. Funct. Bioinf. 2014, 82 (8), 1553-1562.]. While H3 is a critical determinant of antigen recognition, most of its solvent-exposed region does not form direct interactions with the framework region which is the target of humanization. Therefore, it was hypothesized by the present inventors that despite the stringent accuracy that atomistic design calculations demand, Fv model structures may be sufficiently accurate for the purposes of humanization design, according to some embodiments of the present invention.

Modeling accuracy is high and almost equivalent among several modern Fv structure-prediction methods. The AbPredict method was chosen to provide the starting model [Lapidoth, G.; Parker, J.; Prilusky, J.; Fleishman, S. J., AbPredict 2: A Server for Accurate and Unstrained Structure Prediction of Antibody Variable Domains. Bioinformatics 2019, 35 (9), 1591-1593.]. Unlike most other structure-prediction methods which rely on sequence homology, AbPredict relies on energy. Therefore, the resulting model structures are stereochemically and energetically relaxed, mitigating the risk that strain due to modeling artifacts and inaccuracy would lead the humanization workflow to select designs that relieve that artificial strain rather than ones that actually stabilize the CDRs.

All CDR grafting calculations started from the lowest-energy AbPredict model. From this point, all design calculations are identical to the CUMAB protocol above, except that modeling uncertainty is accounted for by including humanized conformations regardless of deviations from the starting model structure.

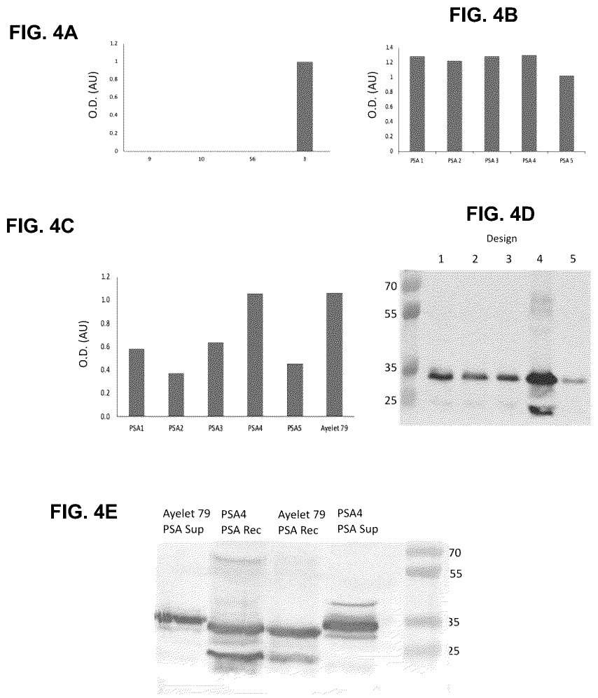

To test CUMAB's ability to humanize antibodies without recourse to experimentally determined structures, an antibody that was raised in mice immunized against human prostate-specific antigen (PSA) was targeted. As with the anti-QSOX1 antibody above, this antibody failed to express in HEK293 cells when its Fv was fused to a human IgG1 constant domain (FIG. 4A; SEQ ID Nos. (heavy/light): 84/83, 86/85, 88/87, 81/82). By contrast, all five top-ranked designs afforded by the method provided herein expressed well in HEK293 cells (FIG. 4B; SEQ ID Nos. (heavy/light): 10/11, 10/12, 13/14, 15/16, and 13/16), were purified using protein G affinity columns, and were tested for binding of recombinantly produced PSA. The afforded designs exhibited some level of binding to PSA, and design 4 (SEQ ID NO: 15/16) exhibited markedly higher levels of binding compared to the others. Since the parental mouse antibody failed to express and be used as a reference, design 4 (SEQ ID NO: 15/16) was compared to a previously isolated antibody that exhibits a dissociation constant (K_{D}) 2-5 nM (FIG. 4C). Immunoprecipitation with recombinant PSA showed that all five designs bound to PSA, with design 4 (SEQ ID NO: 15/16) besting the others (FIG. 4D). In this experimental proof of concept of the method provided herein, the design's affinity for PSA appears as high as the reference antibody's. Finally, design 4 (SEQ ID NO: 15/16) and the reference antibody (Ayelet 79) were tested for their ability to bind the supernatant of LNCaP cells that naturally express PSA, showing that both antibodies bound strongly to natively expressed PSA as well (FIG. 4E). Thus, despite the uncertainty when using Fv models rather than experimentally determined structures, CUMAB produced soluble, high-affinity antibodies in one shot.

### SDR Grafting:

The CDR grafting procedure used in the method provided herein typically raises the sequence identity to the human germline to 80-88% (Table 1). To increase the identity even further, an alternative strategy, called specificity-determining residue (SDR) grafting has been proposed [Kashmiri, S. V. S.; De Pascalis, R.; Gonzales, N. R.; Schlom, J., SDR Grafting—A New Approach to Antibody Humanization. Methods 2005, 36 (1), 25-34; De Pascalis, R.; Iwahashi, M.; Tamura, M.; Padlan, E. A.; Gonzales, N. R.; Santos, A. D.; Giuliano, M.; Schuck, P.; Schlom, J.; Kashmiri, S. V. S., Grafting of “Abbreviated” Complementarity-Determining Regions Containing Specificity-Determining Residues Essential for Ligand Contact to Engineer a Less Immunogenic Humanized Monoclonal Antibody. The Journal of Immunology 2002, 169 (6), 3076-3084].

SDR grafting exchanges only the amino acid positions that directly contact the antigen, whereas the remainder of the antibody, including the CDRs, is humanized. To prevent undesirable changes to the CDR backbone conformation, in SDR grafting, the method used only human germline genes that exhibit CDRs that match the length of the CDRs in the parental antibody, with the exception of H3 (see the Examples section below). Furthermore, since the heavy-chain J gene segment encodes a part of H3, the resulting humanized designs were clustered according to their V gene subgroups as well as their heavy-chain J segment. Thus, unlike in CDR grafting above, designs that differ from one another only in their heavy-chain J segments may be selected. Since SDR grafting demands accurate determination of antigen-binding amino acid positions and due to the uncertainties in modeling the solvent-accessible region in H3, its application has been limited in this exemplary run to experimentally-determined antigen-bound structures.

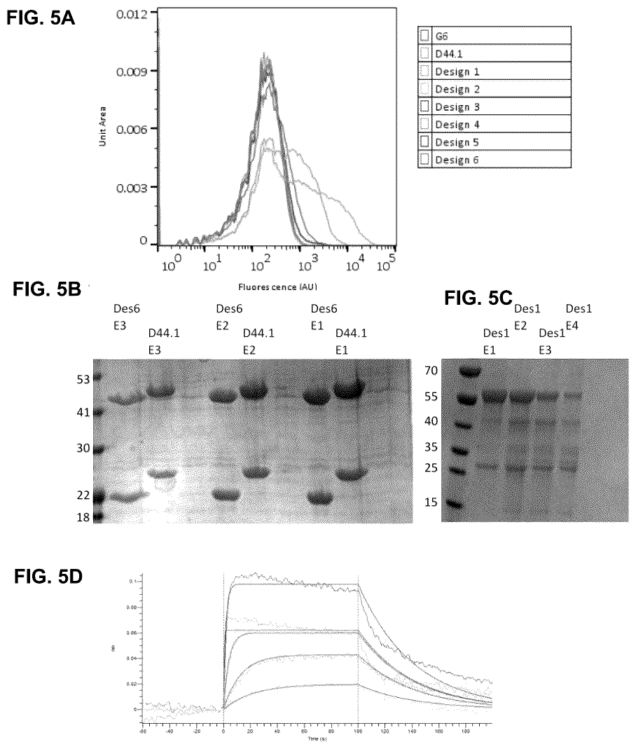

SDR grafting was applied to murine antibody D44.1 (SEQ ID No. 105/104) as observed in its co-crystal structure with hen-egg white lysozyme (PDB entry: 1MLC). In D44.1, 28 positions interact with the antigen out of a total of 63 CDR positions. Thus, in this specific exemplary case, SDR grafting would lead to roughly 90% V gene sequence identity to the human germline in the heavy chain and 93-95% in the light chain compared to roughly 85-87% in the heavy chain and 79-88% in the light chain using the CDR grafting procedure. In an initial experimental screen, six designs (SEQ ID Nos (heavy/light): 27/22, 21/22, 23/24, 25/26, 27/28, and 27/29) were formatted as single-chain variable fragments (scFv) and tested for their binding to lysozyme at 240 nM concentration using yeast cell-surface display (FIG. 5A) [Chao, G.; Lau, W. L.; Hackel, B. J.; Sazinsky, S. L.; Lippow, S. M.; Wittrup, K. D., Isolating and Engineering Human Antibodies Using Yeast Surface Display. Nat. Protoc. 2006, 1 (2), 755-768.]. This preliminary screen indicated that 5 of the 6 designs (SEQ ID Nos (heavy/light): 27/22, 21/22, 23/24, 27/28, and 27/29) exhibited high expression levels, and that design 1 and 6 (SEQ ID Nos (heavy/light): 21/22 and 27/29) bound lysozyme, with design 1 markedly superior to 6. This single-concentration experiment is only qualitative, yet it indicated that design 1 may bind lysozyme more strongly than the parental D44.1 (SEQ ID No. 105/104).

To obtain quantitative binding data, designs 1 and 6 were formatted as human IgG1 antibodies and expressed in addition to D44.1 (expressed as mouse IgG1) in HEK293 cells. The three antibodies expressed well (FIGS. 5B and 5C), and design 1 exhibited 11 nM affinity for lysozyme measured by bilayer interferometry binding experiments (FIG. 5D). Design 1 and D44.1 (SEQ ID Nos. 19/20 and 105/104) were expressed as Fabs (human for design 1 and mouse for D44.1) and design 1 exhibited 41.6 nM affinity while D44.1 exhibited 7.4 nM affinity as measured by surface plasmon resonance. Although there was a slight drop in affinity for design 1, there was a strong increase in humanness and expression.

### Energy-Based Humanization Exploits Non-Homologous Frameworks:

Conventional antibody humanization strategies use the human germline genes that are closest sequence-wise to those of the animal antibody. The energy-based humanization strategy implemented in the herein-provided method, according to some embodiments of the present invention, ignores sequence identity and focuses instead on the structural integrity and energetic stability (structural relaxation) of the humanized designs. The energy-based humanization methodology provided herein expands the scope of potential frameworks from a few dozen high homology ones to thousands. The three test cases presented herein were analyzed to understand what role sequence homology may have in successful antibody humanization.

Strikingly, in all three cases, the experimentally best-performing humanized design does not derive from the best-matched human one according to sequence identity (see, Table 1 in the Examples section below). In both the QSOX1 (SEQ ID No. 59/58) and the PSA exemplary targeting antibodies, neither of the best designs derives from the closest human V gene sub group for either the light or heavy chain, and in the anti-lysozyme antibody, only the heavy chain matches. Furthermore, for both the anti-PSA and anti-lysozyme antibodies, designs based on the same sub group human V genes (designs 3 and 5; SEQ ID Nos: 74/73 and 27/28, respectively) were significantly inferior to the best-performing humanized designs. As an additional comparison with conventional humanization strategies, the anti-QSOX1 antibody (SEQ ID No. 59/58) was subjected to humanization using the “consensus” approach. In this humanization approach, the framework is taken from a sequence-based consensus of V gene subgroups (in this specific case IGKV1 and IGHV4). The designed antibody (SEQ ID NO: 55/56), however, failed to express and its binding to QSOX1 was therefore not tested (FIG. 3F).

Taken together, these results suggest that simply grafting CDRs from an animal antibody onto the closest (or consensus) human germline often fails to recapitulate the animal antibody's stability and functional properties, as has been observed in decades of antibody engineering. By contrast, some of the top-ranked designs from energy-based humanization were well expressed, stable and showed good affinity values, and these typically do not derive from homologous subgroups.

### Method Steps:

Data preparation steps include obtaining a structure of the non-human parental antibody, either an experimentally obtain crystal structure or a calculated/predicted model.

Data preparation steps also include compiling a database of human antibody germline sequences using any available immunogenetics and immunoinformatics source, such as the IMGT reference database.

The crystal or the predicted model is subjected to energy minimization and structure refinement, as this phrase is defined and discussed hereinbelow, to obtain an energetically stabilized and relaxed structure.

The recombination of all representations of each of the four segments, V and J for both light and heavy chains, and for kappa and lambda light chains, is performed separately.

Based on the structure, the residues destined for grafting are identified.

The CDRs of the germline sequence are replaced with the CDRs of the parental antibody.

Any CDR-grafted having post-translational modification motifs and/or extra cysteines are excluded from further analysis; e.g., sequences that have more than two cysteines in either chain or have an Asn-Gly or Asn-X-Ser/Thr motif outside of the CDRs (where X is not Pro).

Each of the CDR-grafted germline sequences is threaded onto the relaxed structure of the parental Ab to thereby obtain a group of humanized Abs.

Each of the threaded humanized Abs is subjected to energy minimization and structural relaxation.

The relaxed humanized Abs are ranked according to their energy score (top-ranked structures have the lowest energy).

Relaxed humanized Ab structures that show a significant backbone conformations deviation (e.g., more than 0.5 Å) in any of the CDRs compared to the parental structure are excluded from further analysis.

The relaxed humanized Abs are clustered into subgroups based on their V gene and J segment affiliation.

Finally, some of the top ranking humanized structures from each cluster are selected for expression, and affinity test with respect to the antigen.

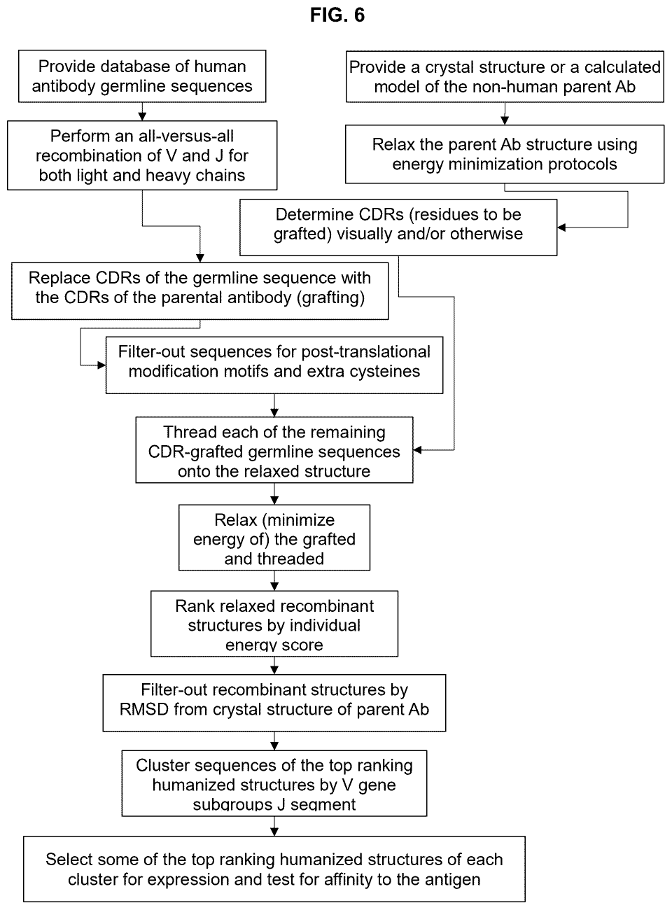

FIG. 6 presents a schematic flowchart of the method provided herein, according to some embodiments of the present invention. As can be seen in FIG. 6, the algorithm starts in two paths, one that provides a database of human antibody germline sequences, and a second which provide a starting structure in the form of a crystal structure or a calculated model of the non-human parent Ab. The two paths converge in the step of thread each of the remaining CDR-grafted germline sequences onto the energy-minimized (relaxed) starting structure. Once the recombinant structures are ranked by individual energy score, and recombinant structures are filter-out by RMSD from the starting structure of the parent Ab, the remaining sequences of the top ranking humanized structures are clustered by V gene subgroups J segment, some of the top ranking humanized structures of each cluster are selected for expression, and the expressed designed are tested for affinity to the antigen.

### Energy Minimization and Structure Refinement:

According to some embodiments, the method presented herein (also referred to as “CUMAb”) makes use of energy minimization and structure refinement to obtain energetically relaxed structures. This structure refinement step is effected for the grafted human antibody structures, and optionally to the structural model of a non-human antibody, also referred to herein as the parent Ab.

Structure refinement is a routine procedure in computational chemistry, and typically involves weight fitting based on free energy minimization, subjected to rules, constraints, and harmonic restraints. The structure refinement step can be effected using any global and/or local energy minimization software based structural constrains and weighted fitting.

According to some embodiments of the present invention, the structural model of a non-human antibody is optionally refined by energy minimization prior to using its coordinates for threading, while optionally fixing the conformations of the CDR residues.

The term “weight fitting”, according to some embodiments of any of the embodiment of the present invention, refers to a one or more computational structure refinement procedures or operations, aimed at optimizing geometrical, spatial and/or energy criteria by minimizing polynomial functions based on predetermined weights, restraints and constrains (constants) pertaining to, for example, sequence homology scores, backbone dihedral angles and/or atomic positions (variables) of the refined structure. According to some embodiments, a weight fitting procedure includes one or more of a modulation of bond lengths and angles, backbone dihedral (Ramachandran) angles, amino acid side-chain packing (rotamers) and an iterative substitution of an amino acid, whereas the terms “modulation of bond lengths and angles”, “modulation of backbone dihedral angles”, “amino acid side-chain packing” and “change of amino acid sequence” are also used herein to refer to, inter alia, well known optimization procedures and operations which are widely used in the field of computational chemistry and biology. An exemplary energy minimization procedure, according to some embodiments of the present invention, is the cyclic-coordinate descent (CCD), which can be implemented with the default all-atom energy function in the Rosetta™ software suite for macromolecular modeling. For a review of general structure optimization and refinement approaches, see for example, “Encyclopedia of Optimization” by Christodoulos A. Floudas and Panos M. Pardalos, Springer Pub., 2008.

According to some embodiments of the present invention, a suitable computational platform for executing the method presented herein, is the Rosetta™ software suite platform, publically available from the “Rosetta@home” at the Baker laboratory, University of Washington, U.S.A., Briefly, Rosetta™ is a molecular modeling software package for understanding protein structures, protein design, protein docking, protein-DNA and protein-protein interactions. The Rosetta software contains multiple functional modules, including RosettaAbinitio, RosettaDesign, RosettaDock, RosettaAntibody, RosettaFragments, RosettaNMR, RosettaDNA, RosettaRNA, RosettaLigand, RosettaSymmetry, and more.

Weight fitting, according to some embodiments, is effected under a set of restraints, constrains and weights, referred to as rules. For example, when refining the backbone atomic positions and dihedral angles of any given polypeptide segment having a first conformation, so as to drive towards a different second conformation while attempting to preserve the dihedral angles observed in the second conformation as much as possible, the computational procedure would use harmonic restraints that bias, e.g., the Ca positions, and harmonic restraints that bias the backbone-dihedral angles from departing freely from those observed in the second conformation, hence allowing the minimal conformational change to take place per each structural determinant while driving the overall backbone to change into the second conformation.

In some embodiments, a global energy minimization is advantageous due to differences between the energy function that was used to determine and refine the source of the template structure, and the energy function used by the method presented herein. By allowing changes to occur in backbone conformation and in rotamer conformation through minimization, the global energy minimization relieves small mismatches and small steric clashes, thereby lowering the total free energy of some template structures by a significant amount.

In some embodiments, energy minimization may include iterations of rotamer sampling (repacking) followed by side chain and backbone minimization. An exemplary refinement protocol is provided in Korkegian, A. et al., Science, 2005. In some embodiments, energy minimization may include more substantial energy minimization in the backbone of the protein.

As used herein, the terms “rotamer sampling” and “repacking” refer to a particular weight fitting procedure wherein favorable side chain dihedral angles are sampled, as defined in the Rosetta software package. Repacking typically introduces larger structural changes to the weight fitted structure, compared to standard dihedral angles minimization, as the latter samples small changes in the residue conformation while repacking may swing a side chain around a dihedral angle such that it occupies an altogether different space in the protein structure.

In some embodiments, wherein the template structure is of a homologous protein, the query sequence is first threaded on the protein's template structure using well established computational procedures. For example, when using the Rosetta software package, according to some embodiments of the present invention, the first two iterations are done with a “soft” energy function wherein the atom radii are defined to be smaller. The use of smaller radius values reduces the strong repulsion forces resulting in a smoother energy landscape and allowing energy barriers to be crossed. The next iterations are done with the standard Rosetta energy function. A “coordinate constraint” term may be added to the standard energy function to allow substantial deviations from the original Ca coordinates. The coordinate constraint term behaves harmonically (Hooke's law), having a weight ranging between about 0.05-0.4 r.e.u (Rosetta energy units), depending on the degree of identity between the query sequence and the sequence of the template structure. During refinement, key residues are only subjected to small range minimization but not to rotamer sampling.

The structure refinement and energy relaxation step, used in the method provided herein, can be effected by the routines provided in, for example, the “Protein Repair One Stop Shop”, or PROSS [Goldenzweig A, Goldsmith M, Hill S E, Gertman O, Laurino P, Ashani Y, Dym O, Unger T, Albeck S, Prilusky J, Lieberman RL, Aharoni A, Silman I, Sussman J L, Tawfik D S, Fleishman S J., Automated Structure- and Sequence-Based Design of Proteins for High Bacterial Expression and Stability. Mol Cell. 2016 Jul. 21; 63 (2): 337-346], and/or FuncLib [Khersonsky O, Lipsh R, Avizemer Z, Ashani Y, Goldsmith M, Leader H, Dym O, Rogotner S, Trudeau D L, Prilusky J, Amengual-Rigo P, Guallar V, Tawfik D S, Fleishman S J. Automated Design of Efficient and Functionally Diverse Enzyme Repertoires. Mol Cell. 2018 Oct. 4; 72 (1): 178-186].

The same tools that are used for energy minimization and structural refinement can be used for ranking the refined structures according to their individual port-refinement (final) minimized energy scoring.

### CONCLUDING REMARKS

Antibody humanization is a foundational technology that has been applied to dozens of antibodies as a necessary step before their clinical use. Despite its importance, however, humanization often leads to a significant reduction in antibody expression yields, stability, affinity, or specificity. Thirty years ago, Foote and Winter concluded that amino acid positions in the framework (vernier positions) are critical for the structural and energetic integrity of the Fv and must be considered for successful humanization. This understanding led to the establishment of an iterative heuristic in which the initially humanized antibody is mutated in vernier positions to identities in the parental antibody to recapitulate the animal antibody's binding or expression properties.

Instead of using only a few dozen homologous frameworks as in the majority of conventional humanization strategies, the method provided herein uses essentially all combinations of possible human gene segments. Although genes belonging to a single subgroup are similar to one another, they contain mutations, including in vernier positions, which may stabilize the specific CDRs of the parental antibody. The results obtained using the herein-provided method demonstrate that selecting the lowest-energy designs from this large space of possible frameworks leads to large gains in stability, expression yields, and binding affinity relative homology-based antibody humanization strategies. Remarkably, CUMAB may start directly from an antibody sequence and use existing software to model the Fv structure. This capability opens the way to humanizing large sets of antibodies obtained through animal immunization without requiring crystallographic analysis. The fundamental insight that antibody stability and activity are determined both by the CDRs and by the amino acid positions on which the CDRs rest may help address other important challenges in antibody engineering to develop general and automated design strategies.

It is expected that during the life of a patent maturing from this application many relevant computational methods for humanizing antibodies will be developed and the scope of the phrase “a computational method for humanizing antibodies” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### Computational Methods

### Building a Database of Antibody Germline Sequences:

A database of antibody germline sequences was afforded by retrieving antibody germline sequences from the IMGT reference database17 (downloaded Jul. 29, 2020). For each gene, only the first allele that was annotated as functional was taken. Additionally, genes had to be annotated as not partial and not reverse complementary. If allele one contains more than two cysteines, a different allele was taken that has two cysteines if possible. This filtering scheme resulted in 54 heavy chain V gene sequences, 6 heavy chain J gene sequences, 39 light chain kappa V gene sequences, 5 light chain kappa J gene sequences, 30 light chain lambda V gene sequences, and 5 light chain kappa J gene sequences.

Thereafter an all-versus-all recombination of these four segments: V and J for both light and heavy chains, and for kappa and lambda light chains, was performed separately, resulting in 63,180 sequences for kappa light chains and 48,600 for lambda light chains.

### Computational CDR Grafting:

CDRs were defined based on combinations of definitions provided elsewhere [MacCallum, R. M.; Martin, A. C.; Thornton, J. M. Antibody-Antigen Interactions: Contact Analysis and Binding Site Topography. J. Mol. Biol. 1996, 262 (5), 732-745; Dondelinger, M.; Filée, P.; Sauvage, E.; Quinting, B.; Muyldermans, S.; Galleni, M.; Vandevenne, M. S. Understanding the Significance and Implications of Antibody Numbering and Antigen-Binding Surface/Residue Definition. Front. Immunol. 2018, 9, 2278] and visual inspection of antibody crystal structures. Starting from a published atomic structure (source from the PDB) of the parental antibody, HMMer was used to identify the segments of the sequence corresponding to the variable region and classify the light chain as kappa or lambda. For each germline sequence corresponding to the light chain classification, the CDRs of the germline sequence are replaced with the CDRs of the parental antibody. Any sequence that contains an Asn-Gly or Asn-X-Ser/Thr (where X is not pro) outside of the CDRs was removed, resulting in more than 20,000 unique sequences per one parental antibody.

### Energy Ranking of CDR-Grafted Sequences:

In cases where a crystal structure is provided, as a first step, the crystal structure of the parental antibody is relaxed through cycles of sidechain and harmonically constrained backbone minimization and combinatorial sidechain packing in the entire Fv (see Relax.xml RosettaScript38). In cases where a bound structure is provided, residues in the interface between the Fv and the antigen were identified using Rosetta and held fixed during the relaxation (see Interface.xml). If the structure was given in a complex with the antigen, the entire antibody Fv-antigen complex was relaxed.

Each CDR-grafted germline sequence was then threaded onto the relaxed Fv structure and relaxed in the same manner with fewer cycles (see Thread_relax.xml). Sequences were ranked according to all atom energy using the ref2015 score function 21. Any model that has a Ca-carbonyl O RMSD of greater than or equal to 0.5 Å in any of the CDRs was excluded from further consideration. Sequences were clustered according to V gene subgroup as defined by IMGT, meaning that only one sequence was taken from each V gene combination. Sequences were visually inspected and, in some cases, the highest ranking representative for a cluster was replaced with a slightly lower ranking one in order to re-use sequences in different clusters and thus minimize cloning.

When starting from a calculated (in silico) model rather than experimental structure, the pipeline was almost identical, with the only difference being that sequences were not excluded for deviating from the model structure due to uncertainties in modeling.

### Computational SDR Grafting:

The parental antibody was classified as having a kappa or lambda light chain as described above. Rosetta was used to identify residues in the interface between the antibody Fv and the antigen (see interface.xml). Antibody germline sequences were selected using the following criteria: the sequences must have the same CDR length in all CDRs excluding H3. Additionally, the sequences must have an H3 length that is equal to or shorter than the H3 length compared to the parental antibody. If the H3 length is shorter than the parental antibody, a number of residues equal to the difference in length of the two H3s from the parental antibody are inserted into the germline sequence. The germline sequences are then threaded and relaxed as described above. Sequences were clustered according to V gene subgroup and heavy J gene subgroup.

### Example 2

### Scripts, Xml, Flags Files and Command Lines

```
Initial Refinement:
  Relax.xml
  <ROSETTASCRIPTS>
          <SCOREFXNS>
                  <ScoreFunction name=“ref2015_full” weights=“ref2015”>
                    <Reweight scoretype=“coordinate_constraint” weight=“0.6”/>
                    <Reweight scoretype=“res_type_constraint” weight=“0.4”/>
                  </ScoreFunction>
                  <ScoreFunction name=“ref2015_soft_full” weights=“ref2015_soft”>
                    <Reweight scoretype=“coordinate_constraint” weight=“0.6”/>
                    <Reweight scoretype=“res_type_constraint” weight=“0.4”/>
                  <ScoreFunction name=“ref2015_no_pssm” weights=“ref2015”>
                    <Reweight scoretype=“coordinate_constraint” weight=“0.6”/>
                  </ScoreFunction>
                  <ScoreFunction name=“ref2015_pure” weights=“ref2015”/>
          </SCOREFXNS>
          <TASKOPERATIONS>
                  <InitializeFromCommandline name=“init”/>
                  <RestrictToRepacking name=“rtr”/>
              <PreventResidues FromRepacking name =“interface”
residues=“%%interface_residues%%”/>
          </TASKOPERATIONS>
          <MOVERS>
                  <PackRotamersMover name=“soft_repack” scorefxn=“ref2015_soft_full”
task_operations=“init,rtr,interface”/>
                  <PackRotamersMover name=“hard_repack” scorefxn=“ref2015_full”
task_operations=“init,rtr,interface”/>
                  <RotamerTrialsMinMover name=“RTmin” scorefxn=“ref2015_Full”
task_operations=“init,rtr,interface”/>
                  <MinMover name=“soft_min” scorefxn=“ref2015_soft_full” chi=“1” bb=“1”
jump=“ALL”/>
                  <MinMover name=“hard_min” scorefxn=“ref2015_full” chi=“1” bb=“1 “
jump=“ALL”/>
                  <AddConstraintsToCurrentConformationMover name=“add_CA_cst”/>
            <AtomTreename=“fold_tree” docking_ft=“1”/>
                  <Prepack name=“prepack” jump_number=“0” scorefxn=“ref2015_no_pssm”/>
                  <ParsedProtocol name=“refinement_block”> #10 movers
                    <Add mover_name=“soft_repack”/>
                    <Add mover_name=“soft_min”/>
                    <Add mover_name=“soft_repack”/>
                    <Add mover_name=“hard_min”/>
                    <Add mover_name=“hard_repack”/>
                    <Add mover_name=“hard_min”/>
                  <Add mover_name=“hard_repack”/>
                    <Add mover_name=“RTmin”/>
                    <Add mover_name=“RTmin”/>
                    <Add mover_name=“hard_min”/>
                  </ParsedProtocol>
                  <LoopOver name=“iter4” mover_name=“refinement_block” iterations=“4”/>
          </MOVERS>
          <FILTERS>
                  <ScoreType name=“stability_score_full” scorefxn=“ref2015_full”
score_type=“total_score” confidence=“0” threshold=“0”/>
                  <ScoreType name=“stability_without_pssm” scorefxn=“ref2015_no_pssm”
score_type=“total_score” confidence=“0” threshold=“0”/>
                  <ScoreType name=“stability_pure” scorefxn=“ref2015_pure”
score_type=“total_score” confidence=“0” threshold=“0”/>
                  <Rmsd name=“rmsd” confidence=“0”/>
                  <Time name=“timer”/>s
          </FILTERS>
          <PROTOCOLS>
                  <Add filter_name=“timer”/>
                  <Add mover_name=“add_CA_cst”/>
              <Add mover_name=“fold_tree”/>
              <Add mover_name=“prepack”/>
                  <Add mover_name=“iter4”/>
                  <Add filter_name=“stability_score_full”/>
                  <Add filter_name=“stability_without_pssm”/>
                  <Add filter_name=“stability_pure”/>
                  <Add filter_name=“rmsd”/>
                  <Add filter_name=“timer”/>
          </PROTOCOLS>
  </ROSETTASCRIPTS>
  Relax.flags
  -use_input_sc
  -extrachi_cutoff 5
  -ignore_unrecognized_res
  -use_occurrence_data
  -chemical:exclude_patches LowerDNA UpperDNA Cterm_amidation SpecialRotamer VirtualBB
ShoveBB Virtual DNAPhosphate VirtualNTerm CTermConnect sc_orbitals pro_hydroxylated_case1
pro_hydroxylated_case2 ser_phosphorylated thr_phosphorylated tyr_phosphorylated tyr_sulfated
lys_dimethylated lys_monomethylated lys_trimethylated lys_acetylated glu_carboxylated c
  ys_acetylated tyr_diiodinated N_acetylated C_methylamidated MethylatedProteinCterm
  -linmem_ig 10
  -ignore_zero_occupancy false
  -nstruct 1
  -mute all
Interface Selection:
  Interface.xml
  <ROSETTASCRIPTS>
    <SCOREFXNS>
      <ScoreFunction name=“ref” weights=“ref2015”/>
    </SCOREFXNS>
    <RESIDUE_SELECTORS>
        <Chain name=“chA” chains=“A”/>
        <Chain name=“chB” chains=“B”/>
        <Chain name=“chC” chains=“C”/>
        <Or name =“chains” selectors =“chA,chB”/>
        <InterfaceByVector name=“interface” cb_dist_cut=“11.0”
          nearby_atom_cut=“5.5” vector_angle_cut=“75. 0”
          vector_dist_cut=“9.0” grp1_selector=“chains”
          grp2_selector=“chC”/>
        <And name=“interface_and_chains” selectors = “interface,chains”/>
        <Not name=“not_interface” selector=“interface_and_chains”/>
    </RESIDUE_SELECTORS>
    <TASKOPERATIONS>
      <InitializeFromCommandline name=“init”/>
      <OperateOnResidueSubset name=“restrict” selector=“not_interface”>
      <PreventRepackingRLT/>
      </OperateOnResidueSubset>
    </TASKOPERATIONS>
    <MOVERS>
    </MOVERS>
    <FILTERS>
      <DesignableResidues name=“designables” task operations=“restrict” designable=“1”/>
    </FILTERS>
    <PROTOCOLS>
      <Add filter_name=“designables”/>
    </PROTOCOLS>
  </ROSETTASCRIPTS>
Thread and Relax:
  Thread_relax.xml
  <ROSETTASCRIPTS>
          <SCOREFXNS>
                  <ScoreFunction name=“ref2015_full” weights=“ref2015”>
                    <Reweight scoretype=“coordinate_constraint” weight=“0.6”/>
                    <Reweight scoretype=“res_type_constraint” weight=“0.4”/>
                  </ScoreFunction>
                  <ScoreFunction name=“ref2015_soft_full” weights=“ref2015_soft”>
                    <Reweight scoretype=“coordinate_constraint” weight=“0.6”/>
                    <Reweight scoretype=“res_type_constraint” weight=“0.4”/>
                  </ScoreFunction>
                  <ScoreFunction name=“ref2015_res_type_cst” weights=“ref2015”>
                    <Reweight scoretype=“res_type_constraint” weight=“0.2”/>
                  </ScoreFunction>
          </SCOREFXNS>
          <RESIDUE_SELECTORS>
                  <Chain name=“chA” chains=“A”/>
                  <Chain name=“chB” chains=“B”/>
                  <InterfaceByVector name=“interface_selector” grp1_selector=“chA”
grp2_selector=“chB”/>
          </RESIDUE_SELECTORS>
          <TASKOPERATIONS>
                    <InitializeFromCommandline name=“init”/>
                    <RestrictToRepacking name=“rtr”/>
                    <ThreadSequence name=“threadseq” target_sequence=“%%Sequence%%”
start_res=“1” allow_design_around=“False”/>
                    <RestrictToInterface name=“interface_acc” jump=“1” distance=“8.0”/>
                    <PreventResiduesFromRepacking name =“interface”
residues=“%%interface_residues%%”/>
          </TASKOPERATIONS>
    <MOVERS>
                <PackRotamersMover name=“soft_repack_thread” scorefxn=“ref2015_soft_full”
task_operations=“init,threadseq,interface”/>
          <PackRotamersMover name=“hard_repack_thread” scorefxn=“ref2015_full”
task_operations=“init,threadseq,interface”/>
                  <TaskAwareCsts name=“add_CA_cst”/>
                  <MinMover name=“soft_min” scorefxn=“ref2015_soft_full” chi=“1” bb=“1”
jump=“1”/>
          <MinMover name=“hard_min” scorefxn=“ref2015_full” chi=“1” bb=“1” jump=“1”/>
      <AtomTree name=“fold_tree” docking_ft=“1”/>
      <PackRotamersMover name=“soft_repack” scorefxn=“ref2015_soft_full”
task_operations=“init,rtr,interface”/>
      <PackRotamersMover name=“hard_repack” scorefxn=“ref2015_full”
task_operations=“init,rtr,interface”/>
      <RotamerTrialsMinMover name=“RTmin” scorefxn=“ref2015_full”
task_operations=“init,rtr,interface”/>
      <ParsedProtocol name=“refinement_block”> #10 movers
          <Add mover_name=“soft_repack”/>
          <Add mover_name=“soft_min”/>
          Add mover_name=“soft_repack”/>
          <Add mover_name=“hard_min”/>
          <Add mover_name=“hard_repack”/>
          <Add mover_name=“hard_min”/>
          <Add mover_name=“hard_repack”/>
          <Add mover_name=“RTmin”/>
          <Add mover_name=“RTmin”/>
          <Add mover_name=“hard_min”/>
      </ParsedProtocol>
      <LoopOver name=“iter4” mover_name=“refinement_block” iterations=“2”/>
                  RotamerTrialsMinMover name=“RTmin” scorefxn=“ref2015_full”
task_operations=“init,rtr,threadseq”/>
    </MOVERS>
          <FILTERS>
                  <CavityVolume name=“a_cavity_vol” confidence=“0”/> SJF: should be less than
or equal to 170A\hat{ }3
                  <ScoreType name=“total_score” scorefxn=“ref2015_res_type_cst”
score_type=“total_score” threshold=“10000.0” confidence=“0”/>
                  <Ddg name=“a_ddg” confidence=“0” scorefxn=“ref2015_res_type_cst”
repeats=“1” threshold=“−15”/>
                  <Sasa name=“a_sasa” confidence=“0” threshold=“1200” />
                  <ShapeComplementarity name=“a_sc” confidence=“0” min_sc=“0.55” />
                  <PackStat name=“a_packstat” confidence=“0” threshold=“0.55” /> LK: 0.66 was
too restrictive
                  <Rmsd name=“rmsd” confidence=“0”/>
                  <Time name=“timer”/>
      <AtomicContactCount name=“a_atomic_contacts_in_interface” partition=“none”
task_operations=“interface_acc” confidence=“0”/> SJF should be >50
      <AtomicContactCount name=“a_atomic_contacts_total” partition=“none” confidence=“0”/>
SJF should be >50
      <PeptideInternalHbondsFilter name=“a_H_bonds_interface”
residue_selector=“interface_selector” confidence=“0”/>
          </FILTERS>
          <PROTOCOLS>
                  <Add filter=“timer”/>
                  <Add mover=“add_CA_cst”/>
      <Add mover=“soft_repack_thread”/>
      <Add mover=“fold_tree”/>
                  <Add mover=“hard_min”/>
                  <Add mover=“hard_repack_thread”/>
                  <Add mover=“hard_min”/>
      <Add mover_name=“iter4”/>
                  <Add filter=“a_ddg”/>
                  <Add filter=“a_sasa”/>
                  <Add filter=“a_sc”/>
                  <Add filter=“a_packstat”/>
                  <Add filter=“a_cavity_vol”/>
                  <Add filter=“a_atomic_contacts_in_interface”/>
      <Add filter=“a_atomic_contacts_in_interface”/>
      <Add filter=“a_atomic_contacts_total”/>
      <Add filter=“a_H_bonds_interface”/>
                  <Add filter=“timer”/>
          </PROTOCOLS>
  </ROSETTASCRIPTS>
```

### Command Lines:

1) {rosetta_exec} -parser: protocol Relax.xml -s {input.pdb} @Relax.flags t -parser: script_vars interface_residues=“interface_residues”
2) {rosetta_exec} -s {input.pdb} -parser: protocol Interface.xml|grep protocols.protein_interface_design.filters.DesignableResiduesFilter>“Interface_residues.txt”
3) {rosetta_exec} -parser: protocol Thread_Relax.xml -s {input.pdb} @Relax.flags -parser: script_vars Sequence={seq} -parser:script_vars interface_residues=“interface_residues”

### Example 3

Sequence identity between parental antibodies (first in each block) and designs to the human germline are presented in Table 1 below.

TABLE 1

| | Closest Heavy V | Closest Light V |
|---|---|---|
| Antibody | Germline Match | Germline Match |
| 492 | IGHV4-4 (57%) | IGKV1-33 (66%) |
| (anti-QSOX1) | | |
| H2b-K4 | IGHV2-5 (86%) | IGKV4-1 (81%) |
| H3-K4 | IGHV3-33 (79%) | IGKV4-1 (81%) |
| Consensus H4-K1 | IGHV4-4 (85%) | IGKV1-NL1 (84%) |
| Clone 10 | IGHV1-46 (61%) | IGKV4-1 (64%) |
| (anti-PSA) | | |
| Design 1 | IGHV1-46 (84%) | IGKV1-NL1 (83%) |
| Design 2 | IGHV1-46 (84%) | IGKV2D-29 (80%) |
| Design 3 | IGHV1-69-2 (88%) | IGKV4-1 (83%) |
| Design 4 | IGHV4-4 (85%) | IGKV1-NL1 (82%) |
| Design 5 | IGHV1-69-2 (88%) | IGKV1-NL1 (82%) |
| D44.1 | IGHV1-46 (67%) | IGKV3-15 (64%) |
| (anti-lysozyme) | | |
| Design 1 | IGHV1-69-2 (90%) | IGKV1-39 (96%) |
| Design 2 | IGHV1-69 (90%) | IGKV1-39 (96%) |
| Design 3 | IGHV1-69-2 (90%) | IGKV1-6 (93%) |
| Design 4 | IGHV1-69 (90%) | IGKV1-9 (96%) |
| Design 5 | IGHV1-69-2 (90%) | IGKV3-15 (97%) |
| Design 6 | IGHV1-69-2 (90%) | IGKV6-21 (96%) |

Table 2 below presents the sequences of the light and heavy chains of the construct, clone and designs 1-5.

TABLE 2

| Construct | Light Chain Variable Sequence | Heavy Chain Variable Sequence |
|---|---|---|
| Clone | SIVMTQTPKILPVSAGDRVTMTCKASQSVVNNVVWY | EVQLQESGPELVKPGASVMISCKTSGYSFTDYNMNWVKQNN |
| 10 | LQKPGQSPKLLIYYASNRYTGVPDRFTGSGSGTDFT | GKSLDWIGVISPDYGTPSYNQKFKGKATLTVDQSSSTAYME |
| | FTISSVQVEDLTVYFCQQHYTSPLTFGAGTKLELK | LNSLTSEDSAVYYCARGKGNYVVFAYWGQGTLVTVAA |
| | (SEQ ID No. 30) | (SEQ ID No. 36) |
| Design | DIQMTQSPSSLSASVGDRVTITCKASQSVVNNVVWY | QVQLVQSGAEVKKPGSSVKVSCKASGYSFTDYNMNWVRQAP |
| 1 | QQKPGKAPKLLIYYASNRYSGVPSRFSGSGSGTDFT | GQGLEWIGVISPDYGTPSYAQKFQGRVTITADESTSTAYME |
| | LTISSLOPEDFATYYCQQHYTSPLTFGPGTKVDIK | LSSLRSEDTAVYYCARGKGNYVVFAYWGQGTLVTVSS |
| | (SEQ ID No. 31) | (SEQ ID No. 37) |
| Design | DIVMTQTPLSLSVTPGQPASISCKASQSVVNNVVWY | QVQLVQSGAEVKKPGSSVKVSCKASGYSFTDYNMNWVRQAP |
| 2 | LQKPGQPPQLLIYYASNRYSGVPDRFSGSGSGTDFT | GQGLEWIGVISPDYGTPSYAQKFQGRVTITADESTSTAYME |
| | LKISRVEAEDVGVYYCQQHYTSPLTFGPGTKVDIK | LSSLRSEDTAVYYCARGKGNYVVFAYWGQGTLVTVSS |
| | (SEQ ID No. 32) | (SEQ ID No. 38) |
| Design | DIVMTQSPDSLAVSLGERATINCKASQSVVNNVVWY | EVQLVQSGAEVKKPGATVKISCKVSGYSFTDYNMNWVQQAP |
| 3 | QQKPGQPPKLLIYYASNRYSGVPDRFSGSGSGTDFT | GKGLEWIGVISPDYGTPSYAEKFQGRVTITADTSTDTAYME |
| | LTISSLQAEDVAVYYCQQHYTSPLTFGPGTKVDIK | LSSLRSEDTAVYYCARGKGNYVVFAYWGQGTLVTVSS |
| | (SEQ ID No. 33) | (SEQ ID No. 39) |
| Design | DIQMTQSPSSLSASVGDRVTITCKASQSVVNNVVWY | QVQLQESGPGLVKPSETLSLTCTVSGYSFTDYNMNWIRQPP |
| 4 | QQKPGKAPKLLIYYASNRYSGVPSRFSGSGSGTEFT | GKGLEWIGVISPDYGTPSYNPSLKSRVTISVDTSKNQFSLK |
| | LTISSLQPEDFATYYCQQHYTSPLTFGPGTKVDIK | LSSVTAADTAVYYCARGKGNYVVFAYWGQGTLVTVSS |
| | (SEQ ID No. 34) | (SEQ ID No. 40) |
| Design | DIQMTQSPSSLSASVGDRVTITCKASQSVVNNVVWY | EVQLVQSGAEVKKPGATVKISCKVSGYSFTDYNMNWVQQAP |
| 5 | QQKPGKAPKLLIYYASNRYSGVPSRFSGSGSGTEFT | GKGLEWIGVISPDYGTPSYAEKFQGRVTITADTSTDTAYME |
| | LTISSLQPEDFATYYCQQHYTSPLTFGPGTKVDIK | LSSLRSEDTAVYYCARGKGNYVVFAYWGQGTLVTVSS |
| | (SEQ ID No. 35) | (SEQ ID No. 41) |

### Example 4

The following example follows the concentration of dissolved oxygen as a measure of QSOX1 activity.

A Clark type oxygen electrode was used to monitor changes in dissolved oxygen concentration as a measure of QSOX1 activity. Antibody was mixed with QSOX1, and reactions were initiated by injection of the model substrate dithiothreitol (DTT). QSOX1 and DTT were at fixed concentrations of 25 nM and 200 μM, respectively, and the antibody concentration was varied. The initial slope of dissolved oxygen concentration was recorded for each antibody concentration. Reactions were performed in duplicate, and the results for each antibody concentration were averaged. Relative activity compared to the uninhibited reaction was plotted against antibody concentration and fitted to the Morrison Ki equation for a tight binding competitive inhibitor, to yield the inhibitory constant (Ki):

$$\frac{Vi}{Vo} = 1 - \frac{( [ E ] + [ I ] + {K}_{i}^{app} ) - \sqrt{{( [ E ] + [ I ] + {K}_{i}^{app} )}^{2} - 4 [ E ] [ I ]}}{2 [ E ]}$$

$${K}_{i}^{app} = {K}_{i} ( 1 + \frac{[ S ]}{{K}_{m}} )$$

Measurements were performed at 25° C. in 50 mM potassium phosphate buffer, pH 7.5, 65 mM NaCl, and 1 mM ethylenediaminetetraacetic acid (EDTA).

The humanized antibodies tested are summarized in Table 3.

TABLE 3

| | Heavy chain | Light Chain |
|---|---|---|
| Ab name | (SEQ ID NO) | (SEQ ID NO) |
| H3newK2 | 4 | 6 |
| H3newK4 | 4 | 8 |
| H3K4 | 3 | 8 |
| H2bK4 | 2 | 8 |

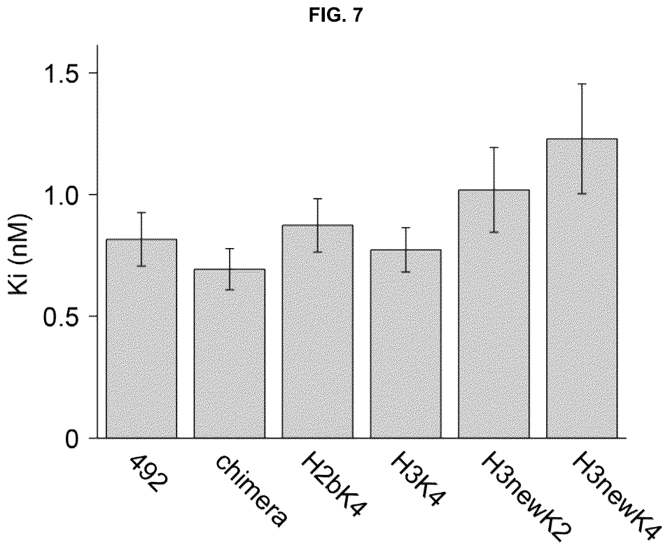

FIG. 7 presents a comparative bar-graph, comparing the activity (affinity) assay results conducted for the expressed antibodies 492 (SEQ ID Nos. (heavy/light): 79/80), chimera, h2bK4 (SEQ ID Nos.: 2/8), h3K4 (SEQ ID Nos.: 3/8), h3newK2 (SEQ ID Nos.: 4/6) and h3newK4 (SEQ ID Nos.: 4/8).

As can be seen in FIG. 7, the humanized antibodies (h2bK4, h3K4, h3newK2 and h3newK4; Table 3) have sub-nanomolar Ki values, similar to the original murine antibody MAb492 (SEQ ID Nos. (heavy/light): 79/80).

Table 4 presents fifteen anti-QSOX1designs which were tested experimentally.

TABLE 4

| | Heavy | SEQ ID | Light | SEQ ID |
|---|---|---|---|---|
| Design | chain | NO: | chain | NO: |
| H2a-K1a | H2a | 91 | K1a | 94 |
| H2a-K1b | | | K1b | 98 |
| H2a-K2 | | | K2 | 95 |
| H2a-K3 | | | K3 | 96 |
| H2a-K4 | | | K4 | 97 |
| H2b-K1a | H2b | 92 | K1a | 94 |
| H2b-K1b | | | K1b | 98 |
| H2b-K2 | | | K2 | 95 |
| H2b-K3 | | | K3 | 96 |
| H2b-K4 | | | K4 | 97 |
| H3-K1a | H3 | 93 | K1a | 94 |
| H3-K1b | | | K1b | 98 |
| H3-K2 | | | K2 | 95 |
| H3-K3 | | | K3 | 96 |
| H3-K4 | | | K4 | 97 |

### Example 5

The abovementioned target for humanization, murine monoclonal antibody that inhibits the enzymatic activity of human Quiescin Sulfhydryl Oxidase 1 (QSOX1) is an antibody under development as a potential cancer therapeutic due to its ability to block the contribution of QSOX1 to extracellular matrix support of tumor growth and metastasis (pubmed ID: 32064042). This antibody is a stringent test for humanization because chimerizing its mouse Fv with a human IgG1 constant region leads to a complete loss of expressibility in HEK293 cells23.

This challenge was previously addressed using the AbLIFT method, which uses atomistic design calculations to improve the molecular interactions between the Fv light and heavy domains [Warszawski, S. et al. Optimizing antibody affinity and stability by the automated design of the variable light-heavy chain interfaces. PLOS Comput. Biol. 15, e1007207 (2019).]. One of the designs, AbLIFT18, rescued expression in HEK293 cells and QSOX1 inhibition of the chimeric antibody composed of human IgG1 constant regions and murine Fv. However, this design was an incompletely humanized antibody (66.3% and 57.3% V gene sequence identity to the nearest human germline in the light and heavy chains, respectively), meaning that it was not suitable for therapeutic applications in humans.

To simulate a realistic humanization scenario, the present inventors started directly from the parental mouse antibody and used AbLIFT18 and the mouse parental antibody as controls. The present inventors ordered genes encoding the five top-ranked CUMAb designs formatted as separate light and heavy chains and experimentally tested all 15 unique pairs of light and heavy chains from among the top-ranked five designs. Remarkably, 12 pairs showed comparable expression levels on a dot-blot analysis relative to AbLIFT18, while no detectable expression was detected for the chimeric construct comprising the mouse Fv and human constant domains. Furthermore, electrophoretic-mobility analysis after purification revealed that seven designs showed comparable expression levels to AbLIFT18 without obvious misfolding or aggregation.

The expressible designs were purified and screened for QSOX1 inhibition. The two most successful designs exhibited similar inhibition constants to that of the parental antibody. These designs share the same light chain and exhibit 81.2% V gene sequence identity to the nearest human germline gene, and 79.4 and 85.7% identities in the heavy chain. These V gene sequence identities are significantly higher than those for the mouse antibody and AbLIFT18, which both have 66.3% sequence identity (light chain) 57.3% (heavy chain). Additionally, these V gene sequence identities are in the range of those among FDA-approved humanized antibodies, which have a mean of 84% sequence identity in the light chain and 81% in the heavy chain. Thus, the two designs recapitulated the parental mouse expression levels and activity using a human Fv framework and without requiring back mutations.

The present inventors measured the melting temperatures of the two designs as well as the parental antibody with nano-differential scanning fluorimetry (nano-DSF) and found all three to be above 70° C. Interestingly, the two designs derive from different heavy chain V gene subgroups and have 36 mutations between them. Due to these mutations, they have strikingly different patterns of surface charge.

Thus, CUMAb produces antibodies that are functionally nearly identical antibodies but have very different surface properties. As surface properties have been associated with changes in the propensity of antibodies to self-associate or form non-specific interactions, it may be very advantageous to have multiple humanized options of an antibody with different surface properties.

The present inventors determined a co-crystal structure of one of the best-performing designs and the oxidoreductase fragment of human QSOX1 and found that the design and parental antibody are strikingly similar, with only a 0.75 Å c-alpha RMSD between them despite 51 mutations between the parental and humanized antibodies. These results verify CUMAb's atomic accuracy and its ability to rapidly produce functionally similar yet more stable humanized designs even in a case that defied previous humanization efforts.

Table 5 presents sequence identity between parental antibodies (first in each block) and designs to the human germline. % identity computed using IgBLAST.

TABLE 5

| | Closest Heavy V | Closest Light V |
|---|---|---|
| Antibody | Germline Match | Germline Match |
| mαQSOX1 | IGHV4-4 (57%) | IGKV1-33 (66%) |
| hαQSOX1.1 | IGHV3-33 (79%) | IGKV4-1 (81%) |
| (SEQ ID NO. 3/8) | | |
| hαQSOX1.2 | IGHV2-5 (86%) | IGKV4-1 (81%) |
| (SEQ ID NO. 2/8) | | |
| Consensus H4-K1 | IGHV4-4 (85%) | IGKV1-NL1 (84%) |
| mαAXL | IGHV3-23 (78.6%) | IGKV1-16 (62.1%) |
| hαAXL | IGHV3-23 (88.8%) | IGKVID-13 (84.2%) |
| (SEQ ID NOs. 17/18) | | |
| mαHEWL | IGHV1-46 (67%) | IGKV3-15 (64%) |
| hαHEWL | IGHV1-69-2 (90%) | IGKV1-39 (96%) |
| (SEQ ID NOs. 19/20) | | |

The sequences of the designs reported in this example are listed in Table 6 below, such as, for example, anti-QSOX1 heavy chain H3 (haQSOX1.1) and anti-QSOX1 heavy chain H2b (haQSOX1.2)

### Example 6

The following is a list of antibodies pertinent to the method of designing and preparing a humanized antibody (CUMAb), according to embodiments of the present invention.

TABLE 6

| SEQ | | |
|---|---|---|
| ID | | Identity and |
| NO. | Sequence | comments |
| 1 | MGWSCIILFLVATATGVHCQITLKESGPTLVKPTQTLTLTCTVSGFSLT | Anti-QSOX1 heavy |
| | GYGVNWIRQPPGKALEWLGMIWGDGRTDYSPSLKSRLTITKDTSKNQVV | chain H2a |
| | LTMTNMDPVDTATYYCASDYYGSGSFAYWGQGTTVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 2 | MGWSCIILFLVATATGVHCQITLKESGPTLVKPTQTLTLTCTVSGFSLT | Anti-QSOX1 heavy |
| | GYGVNWIRQPPGKALEWLGMIWGDGRTDYSPSLKSRLTITKDTSKNQVV | chain H2b |
| | LTMTNMDPVDTATYYCASDYYGSGSFAYWGRGTLVTVSSASTKGPSVFP | (hαQSOX1.2) |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 3 | MGWSCIILFLVATATGVHCQVQLVESGGGLVQPGGSLRLSCSVSGFSLT | Anti-QSOX1 heavy |
| | GYGVNWVRQAPGKGLEWLGMIWGDGRTDYADSVKGRFTISRDNSKNTLY | chain H3 |
| | LQMSSLRAEDTAVYYCASDYYGSGSFAYWGQGTLVTVSSASTKGPSVFP | (hαQSOX1.1) |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 4 | MGWSCIILFLVATATGVHCQVQLVESGGGLVQPGRSLRLSCTVSGFSLT | Anti-QSOX1 heavy |
| | GYGVNWFRQAPGKGLEWLGMIWGDGRTDYTASVKGRFTISRDGSKSIAY | chain H3new |
| | LQMNSLKTEDTAVYYCASDYYGSGSFAYWGQGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 5 | MGWSCIILFLVATATGVHSDIQMTQSPSSLSASVGDRVTITCKASQDVS | Anti-QSOX1 light |
| | TAVAWYQQKPGKAPKLLIHSASYRYTGVPSRFSGSGSGTDFTFTISSLQ | chain K1a |
| | PEDIATYYCQQHYSIPLTFGQGTKVEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 6 | MGWSCIILFLVATATGVHSDIVMTQTPLSLSVTPGQPASISCKASQDVS | Anti-QSOX1 light |
| | TAVAWYLQKPGQPPQLLIHSASYRYTGVPDRFSGSGSGTDFTLKISRVE | chain K2 |
| | AEDVGVYYCQQHYSIPLTFGPGTKVDIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 7 | MGWSCIILFLVATATGVHSEIVMTQSPATLSLSPGERATLSCKASQDVS | Anti-QSOX1 light |
| | TAVAWYQQKPGQAPRLLIHSASYRYTGIPARFSGSGSGTDFTLTISSLQ | chain K3 |
| | PEDFAVYYCQQHYSIPLTFGQGTKVEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 8 | MGWSCIILFLVATATGVHSDIVMTQSPDSLAVSLGERATINCKASQDVS | Anti-QSOX1 light |
| | TAVAWYQQKPGQPPKLLIHSASYRYTGVPDRFSGSGSGTDFTLTISSLQ | chain K4 |
| | AEDVAVYYCQQHYSIPLTFGQGTRLEIKRTVAAPSVFIFPPSDEQLKSG | (hαQSOX1.1, |
| | TASVVCLLNNFYPREAKVQWKVDNALQSGNSQESVTEQDSKDSTYSLSS | hαQSOX1.2) |
| | TLTLSKADYEKHKVYACEVTHQGLSSPVTKSFNRGEC | |
| 9 | MGWSCIILFLVATATGVHSAIQLTQSPSSLSASVGDRVTITCKASQDVS | Anti-QSOX1 light |
| | TAVAWYQQKPGKAPKLLIHSASYRYTGVPSRFSGSGSGTDFTLTISSLQ | chain K1b |
| | PEDFATYYCQQHYSIPLTFGQGTKVEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 10 | MDWTWRFLFVVAAATGVQSQVQLVQSGAEVKKPGSSVKVSCKASGYSFT | Anti-PSA Design |
| | DYNMNWVRQAPGQGLEWIGVISPDYGTPSYAQKFQGRVTITADESTSTA | 1,2 heavy chain |
| | YMELSSLRSEDTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 11 | MRVPAQLLGLLLLWLSGARCDIQMTQSPSSLSASVGDRVTITCKASQSV | Anti-PSA Design 1 |
| | VNNVVWYQQKPGKAPKLLIYYASNRYSGVPSRFSGSGSGTDFTLTISSL | light chain |
| | QPEDFATYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 12 | MRVPAQLLGLLLLWLSGARCDIVMTQTPLSLSVTPGQPASISCKASQSV | Anti-PSA Design 2 |
| | VNNVVWYLQKPGQPPQLLIYYASNRYSGVPDRFSGSGSGTDFTLKISRV | light chain |
| | EAEDVGVYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 13 | MDWTWRFLFVVAAATGVQSEVQLVQSGAEVKKPGATVKISCKVSGYSFT | Anti-PSA Design |
| | DYNMNWVQQAPGKGLEWIGVISPDYGTPSYAEKFQGRVTITADTSTDTA | 3,5 heavy chain |
| | YMELSSLRSEDTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 14 | MRVPAQLLGLLLLWLSGARCDIVMTQSPDSLAVSLGERATINCKASQSV | Anti-PSA Design 3 |
| | VNNVVWYQQKPGQPPKLLIYYASNRYSGVPDRFSGSGSGTDFTLTISSL | light chain |
| | QAEDVAVYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 15 | MDWTWRFLFVVAAATGVQSQVQLQESGPGLVKPSETLSLTCTVSGYSFT | Anti-PSA Design 4 |
| | DYNMNWIRQPPGKGLEWIGVISPDYGTPSYNPSLKSRVTISVDTSKNQF | heavy chain |
| | SLKLSSVTAADTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 16 | MRVPAQLLGLLLLWLSGARCDIQMTQSPSSLSASVGDRVTITCKASQSV | Anti-PSA Design |
| | VNNVVWYQQKPGKAPKLLIYYASNRYSGVPSRFSGSGSGTEFTLTISSL | 4,5 light chain |
| | QPEDFATYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 17 | MDWTWRFLFVVAAATGVQSEVQLVESGGGLIQPGGSLRLSCAASGFTFS | Anti-AXL |
| | TYGMSWVRQAPGKGLELVATINGNGGSAYYADSVKGRFTISRDNSKNTL | humanized heavy |
| | YLQMNSLRAEDTAVYYCVRERDYYGNSRYFDVWGQGTMVTVSSASTKGP | chain (hαAXL) |
| | SVFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKEY | |
| | KCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRW | |
| | QQGNVFSCSVMHEALHNHYTQKSLSLSPGK | |
| 18 | MRVPAQLLGLLLLWLSGARCAIQLTQSPSSLSASVGDRVTITCKASQNV | Anti-AXL |
| | DTNVAWYQQKPGKAPKPLIFSAFLRYSGVPSRFSGSGSGTDFTLTISSL | humanized light |
| | QPEDFATYYCQQYNNYPYTFGPGTKVDIKRTVAAPSVFIFPPSDEQLKS | chain (hαAXL) |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 19 | MGWSCIILFLVATATGVHCEVQLVQSGAEVKKPGATVKISCKVSGYTFS | Anti-HEWL Design |
| | TYWMEWVQQAPGKGLEWMGEVLPGSGSTYYAEKFQGRVTITADTSTDTA | 1 heavy chain |
| | YMELSSLRSEDTAVYYCARGDGYFQHWGQGTLVTVSSASTKGPSVFPLA | (hαHEWL) Fab |
| | PSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHHHH | |
| | HH | |
| 20 | MGWSCIILFLVATATGVHSDDIQMTQSPSSLSASVGDRVTITCRASQSI | Anti-HEWL Design |
| | SSNLNWYQQKPGKAPKLLIYYASSLQSGVPSRFSGSGSGTDFTLTISSL | 1 light chain |
| | QPEDFATYYCQQSNSWPRTFGPGTKVDIKRTVAAPSVFIFPPSDEQLKS | (hαHEWL) Fab |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 21 | QVQLVQSGAEVKKPGSSVKVSCKASGYTFSTYWIEWVRQAPGQGLEWMG | Anti-HEWL Design |
| | EILPGSGSAYYAQKFQGRVTITADESTSTAYMELSSLRSEDTAVYYCAR | 2 heavy chain |
| | GDGYFDLWGRGTLVTVSS | variable |
| 22 | DIQMTQSPSSLSASVGDRVTITCRASQSISSNLNWYQQKPGKAPKLLIY | Anti-HEWL Design |
| | YASSLQSGVPSRFSGSGSGTDFTLTISSLQPEDFATYYCQQSNSWPRTF | 1,2 light chain |
| | GPGTKVDIK | variable |
| 23 | EVQLVQSGAEVKKPGATVKISCKVSGYTFSTYWMEWVQQAPGKGLEWMG | Anti-HEWL Design |
| | EVLPGSGSTYYAEKFQGRVTITADTSTDTAYMELSSLRSEDTAVYYCAR | 3 heavy chain |
| | GDGYFDYWGQGTLVTVSS | variable |
| 24 | AIQMTQSPSSLSASVGDRVTITCRASQGISNNLGWYQQKPGKAPKLLIY | Anti-HEWL Design |
| | YASSLQSGVPSRFSGSGSGTDFTLTISSLQPEDFATYYCLQSNSWPRTF | 3 light chain |
| | GPGTKVDIK | variable |
| 25 | QVQLVQSGAEVKKPGSSVKVSCKASGYTFSTYWIEWVRQAPGQGLEWMG | Anti-HEWL Design |
| | EILPGSGSAYYAQKFQGRVTITADESTSTAYMELSSLRSEDTAVYYCAR | 4 heavy chain |
| | GDGWFDSWGQGTLVTVSS | variable |
| 26 | DIQLTQSPSFLSASVGDRVTITCRASQGISSNLAWYQQKPGKAPKLLIY | Anti-HEWL Design |
| | YASTLQSGVPSRFSGSGSGTEFTLTISSLQPEDFATYYCQQSNSWPRTF | 4 light chain |
| | GPGTKVDIK | variable |
| 27 | EVQLVQSGAEVKKPGATVKISCKVSGYTFSTYWMEWVQQAPGKGLEWMG | Anti-HEWL Design |
| | EVLPGSGSTYYAEKFQGRVTITADTSTDTAYMELSSLRSEDTAVYYCAR | 1,5,6 heavy chain |
| | GDGYFQHWGQGTLVTVSS | variable |
| 28 | EIVMTQSPATLSVSPGERATLSCRASQSVSSNLAWYQQKPGQAPRLLIY | Anti-HEWL Design |
| | YASTRATGIPARFSGSGSGTEFTLTISSLQSEDFAVYYCQQSNSWPRTF | 5 light chain |
| | GPGTKVDIK | variable |
| 29 | EIVLTQSPDFQSVTPKEKVTITCRASQSISSNLHWYQQKPDQSPKLLIK | Anti-HEWL Design |
| | YASQSFSGVPSRFSGSGSGTDFTLTINSLEAEDAATYYCHQSNSWPRTF | 6 light chain |
| | GPGTKVDIK | variable |
| 30 | SIVMTQTPKILPVSAGDRVTMTCKASQSVVNNVVWYLQKPGQSPKLLIY | Anti-PSA Clone 10 |
| | YASNRYTGVPDRFTGSGSGTDFTFTISSVQVEDLTVYFCQQHYTSPLTF | Light Chain |
| | GAGTKLELK | variable |
| 31 | DIQMTQSPSSLSASVGDRVTITCKASQSVVNNVVWYQQKPGKAPKLLIY | Anti-PSA Design 1 |
| | YASNRYSGVPSRFSGSGSGTDFTLTISSLQPEDFATYYCQQHYTSPLTF | Light Chain |
| | GPGTKVDIK | variable |
| 32 | DIVMTQTPLSLSVTPGQPASISCKASQSVVNNVVWYLQKPGQPPQLLIY | Anti-PSA Design 2 |
| | YASNRYSGVPDRFSGSGSGTDFTLKISRVEAEDVGVYYCQQHYTSPLTF | Light Chain |
| | GPGTKVDIK | variable |
| 33 | DIVMTQSPDSLAVSLGERATINCKASQSVVNNVVWYQQKPGQPPKLLIY | Anti-PSA Design 3 |
| | YASNRYSGVPDRFSGSGSGTDFTLTISSLQAEDVAVYYCQQHYTSPLTF | Light Chain |
| | GPGTKVDIK | variable |
| 34 | DIQMTQSPSSLSASVGDRVTITCKASQSVVNNVVWYQQKPGKAPKLLIY | Anti-PSA Design 4 |
| | YASNRYSGVPSRFSGSGSGTEFTLTISSLQPEDFATYYCQQHYTSPLTF | Light Chain |
| | GPGTKVDIK | variable |
| 35 | DIQMTQSPSSLSASVGDRVTITCKASQSVVNNVVWYQQKPGKAPKLLIY | Anti-PSA Design 5 |
| | YASNRYSGVPSRFSGSGSGTEFTLTISSLQPEDFATYYCQQHYTSPLTF | Light Chain |
| | GPGTKVDIK | variable |
| 36 | EVQLQESGPELVKPGASVMISCKTSGYSFTDYNMNWVKQNNGKSLDWIG | Anti-PSA Clone 10 |
| | VISPDYGTPSYNQKFKGKATLTVDQSSSTAYMELNSLTSEDSAVYYCAR | Heavy Chain |
| | GKGNYVVFAYWGQGTLVTVAA | variable |
| 37 | QVQLVQSGAEVKKPGSSVKVSCKASGYSFTDYNMNWVRQAPGQGLEWIG | Anti-PSA Design 1 |
| | VISPDYGTPSYAQKFQGRVTITADESTSTAYMELSSLRSEDTAVYYCAR | Heavy Chain |
| | GKGNYVVFAYWGQGTLVTVSS | variable |
| 38 | QVQLVQSGAEVKKPGSSVKVSCKASGYSFTDYNMNWVRQAPGQGLEWIG | Anti-PSA Design 2 |
| | VISPDYGTPSYAQKFQGRVTITADESTSTAYMELSSLRSEDTAVYYCAR | Heavy Chain |
| | GKGNYVVFAYWGQGTLVTVSS | variable |
| 39 | EVQLVQSGAEVKKPGATVKISCKVSGYSFTDYNMNWVQQAPGKGLEWIG | Anti-PSA Design 3 |
| | VISPDYGTPSYAEKFQGRVTITADTSTDTAYMELSSLRSEDTAVYYCAR | Heavy Chain |
| | GKGNYVVFAYWGQGTLVTVSS | variable |
| 40 | QVQLQESGPGLVKPSETLSLTCTVSGYSFTDYNMNWIRQPPGKGLEWIG | Anti-PSA Design 4 |
| | VISPDYGTPSYNPSLKSRVTISVDTSKNQFSLKLSSVTAADTAVYYCAR | Heavy Chain |
| | GKGNYVVFAYWGQGTLVTVSS | variable |
| 41 | EVQLVQSGAEVKKPGATVKISCKVSGYSFTDYNMNWVQQAPGKGLEWIG | Anti-PSA Design 5 |
| | VISPDYGTPSYAEKFQGRVTITADTSTDTAYMELSSLRSEDTAVYYCAR | Heavy Chain |
| | GKGNYVVFAYWGQGTLVTVSS | variable |
| 42 | QVQLKQSGPGLVAPSQSLSITCTVSGFSLTGYGVNWVRQSPGKGLEWLG | 4IJ3 var. heavy, |
| | MIWGDGRTDYKSALKSRLSITKDNSKSQVFLKMNSLQTDDTARYFCASD | region |
| | YYGSGSFAYWGQGTLVTVSA | Murine, not |
| | | designed |
| 43 | DVVMTQSHKFMSTSVGDRVSITCKASQDVSTAVAWYQQKSGQSPKLLIH | 4IJ3 var. light, |
| | SASYRYTGVPDRFTGSGSGTDFTFTISSVQAEDLAVYYCQQHYSIPLTF | region |
| | GAGTKLELK | Murine, not |
| | | designed |
| 44 | MGWSCIILFLVATATGVHCQVQLKQSGPGLVAPSQSLSITCTVSGFSLT | Ch3, heavy, Murine |
| | GYGVNWVRQSPGKGLEWLGMIWGDGRTEYKSALKSRLSITKDNSKSQVF | Fv, human Fc, Fv |
| | LKMNSLQTDDTARYYCASDYYGSGALEYWGQGTLVTVSAASTKGPSVFP | was designed |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 45 | MGWSCIILFLVATATGVHSDVVMTQSHKFMSTSVGDRVSITCKASQDVS | Ch3, light, Murine |
| | TAVAWYQQKSGQAPKLLIHSASYRPTGVPDRFTGSGSGTDFTFTISSVQ | Fv, human Fc, Fv |
| | AEDLAVYYCQQHYSIPLTFGAGTKLELKRTVAAPSVFIFPPSDEQLKSG | was designed |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 46 | MGWSCIILFLVATATGVHCQVQLKQSGPGLVAPSQSLSITCTVSGFSLT | Ch18, heavy, |
| | GYGVNWFRQSPGKGLEWLGMIWGDGRTDYKSALKSRLSITKDNSKSQVF | Murine Fv, human |
| | LKMNSLQTDDTARYFCASDYYGSGSFAYWGQGTLVTVSAASTKGPSVFP | Fc, Fv was |
| | LAPSSKSTSGGTAALGCLVKDYFPEPVTVSWNSGALTSGVHTFPAVLQS | designed |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 47 | MGWSCIILFLVATATGVHSDVVMTQSHKFMSTSVGDRVSITCKASQDVS | Ch18, light, Murine |
| | TAVAWYQQKSGQPPKLLIHSASYRVTGVPDRFTGSGSGTDFTFTISSVQ | Fv, human Fc, Fv |
| | AEDLAVYYCQQHYSIPFTFGAGTKLELKRTVAAPSVFIFPPSDEQLKSG | was designed |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 48 | MGWSCIILFLVATATGVHCQITLKESGPTLVKPTQTLTLTCTFSGFSLT | H2, Human Fc, |
| | GYGVNWIRQPPGKALEWLGMIWGDGRTDYSPSLKSRLTITKDTSKNQVV | humanized Fv |
| | LTMTNMDPVDTATYYCASDYYGSGSFAYWGQGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 49 | MGWSCIILFLVATATGVHCQVQLQESGPGLVKPSETLSLTCTVSGFSLT | H4, Human Fc, |
| | GYGVNWIRQPPGKGLEWLGMIWGDGRTDYNPSLKSRVTISVDTSKNQFS | humanized Fv |
| | LKLSSVTAADTAVYYCASDYYGSGSFAYWGQGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 50 | MGWSCIILFLVATATGVHSDIQMTQSPSSLSASVGDRVTITCKASQDVS | K1a, Human Fc, |
| | TAVAWFQQKPGKAPKLLIHSASYRVTGVPSRFSGSGSGTDFTLTISSLQ | humanized Fv |
| | PEDFATYYCQQHYSIPFTFGPGTKVDIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 51 | MGWSCIILFLVATATGVHSAIQLTQSPSSLSASVGDRVTITCKASQDVS | K1b, Human Fc, |
| | TAVAWYQQKPGKAPKLLIHSASYRVTGVPSRFSGSGSGTDFTLTISSLQ | humanized Fv |
| | PEDFATYYCQQHYSIPFTFGPGTKVDIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 52 | MGWSCIILFLVATATGVHSDIVMTQTPLSLSVTPGQPASISCKASQDVS | K2, Human Fc, |
| | TAVAWYLQKPGQPPQLLIHSASYRVTGVPDRFSGSGSGTDFTLKISRVE | humanized Fv |
| | AEDVGVYYCQQHYSIPFTFGPGTKVDIKRTVAAPSVFIFPPSDEQLKSG | Human Fc, |
| | TASVVCLLNNFYPREAKVQWKVDNALQSGNSQESVTEQDSKDSTYSLSS | humanized Fv |
| | TLTLSKADYEKHKVYACEVTHQGLSSPVTKSFNRGEC | |
| 53 | MGWSCIILFLVATATGVHSEIVMTQSPATLSLSPGERATLSCKASQDVS | K3, Human Fc, |
| | TAVAWYQQKPGQAPRLLIHSASYRVTGIPARFSGSGSGTDFTLTISSLQ | humanized Fv |
| | PEDFAVYYCQQHYSIPFTFGPGTKVDIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 54 | MGWSCIILFLVATATGVHSDIVMTQSPDSLAVSLGERATINCKASQDVS | K4, Human Fc, |
| | TAVAWYQQKPGQPPKLLIHSASYRVTGVPDRFSGSGSGTDFTLTISSLQ | humanized Fv |
| | AEDVAVYYCQQHYSIPFTFGQGTRLEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 55 | MGWSCIILFLVATATGVHCQVQLQESGPGLVKPSETLSLTCTVSGFSLT | H4 consensus, |
| | GYGVNWIRQPPGKGLEWLGMIWGDGRTDYNPSLKSRVTISVDTSKNQFS | Human Fc, |
| | LKLSSVTAADTAVYYCASDYYGSGSFAYWGQGTLVTVSAASTKGPSVFP | humanized Fv |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 56 | MGWSCIILFLVATATGVHSDIQMTQSPSSLSASVGDRVTITCKASQDVS | K1 consensus, |
| | TAVAWYQQKPGKAPKLLIHSASYRYSGVPSRFSGSGSGTDFTLTISSLQ | Human Fc, |
| | PEDFATYYCQQHYSIPLTFGQGTKVELKRTVAAPSVFIFPPSDEQLKSG | humanized Fv |
| | TASVVCLLNNFYPREAKVQWKVDNALQSGNSQESVTEQDSKDSTYSLSS | |
| | TLTLSKADYEKHKVYACEVTHQGLSSPVTKSENRGEC | |
| 57 | MGWSCIILFLVATATGVHCQVQLKQSGPGLVAPSQSLSITCTVSGFSLT | 492, heavy, Murine |
| | GYGVNWVRQSPGKGLEWLGMIWGDGRTDYKSALKSRLSITKDNSKSQVF | Fc, murine Fv |
| | LKMNSLQTDDTARYFCASDYYGSGSFAYWGQGTLVTVSAASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 58 | MGWSCIILFLVATATGVHSDVVMTQSHKFMSTSVGDRVSITCKASQDVS | 492, light, Murine |
| | TAVAWYQQKSGQSPKLLIHSASYRYTGVPDRFTGSGSGTDFTFTISSVQ | Fc, murine Fv |
| | AEDLAVYYCQQHYSIPLTFGAGTKLELKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 59 | MGWSCIILFLVATATGVHCQITLKESGPTLVKPTQTLTLTCTVSGFSLT | H2a, Human Fc, |
| | GYGVNWIRQPPGKALEWLGMIWGDGRTDYSPSLKSRLTITKDTSKNQVV | humanized Fv |
| | LTMTNMDPVDTATYYCASDYYGSGSFAYWGQGTTVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 60 | MGWSCIILFLVATATGVHCQITLKESGPTLVKPTQTLTLTCTVSGFSLT | H2b, Human Fc, |
| | GYGVNWIRQPPGKALEWLGMIWGDGRTDYSPSLKSRLTITKDTSKNQVV | humanized Fv |
| | LTMTNMDPVDTATYYCASDYYGSGSFAYWGRGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 61 | MGWSCIILFLVATATGVHCQVQLVESGGGLVQPGGSLRLSCSVSGFSLT | H3, Human Fc, |
| | GYGVNWVRQAPGKGLEWLGMIWGDGRTDYADSVKGRFTISRDNSKNTLY | humanized Fv |
| | LQMSSLRAEDTAVYYCASDYYGSGSFAYWGQGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 62 | MRVPAQLLGLLLLWLSGARCSIVMTQTPKILPVSAGDRVTMTCKASQSV | Anti-PSA Clone 9 |
| | VNNVVWYLQKPGQSPKLLIYYASNRYTGVPDRFTGSGSGTDFTFTISSV | Light chain, Human |
| | QVEDLTVYFCQQHYTSPLTFGAGTKLELKRTVAAPSVFIFPPSDEQLKS | Fc, mouse Fv |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 63 | MRVPAQLLGLLLLWLSGARCSIVMTQTPKILPVSAGDRVTMTCKASQSV | Anti-PSA Clone 10 |
| | VNNVVWYLQKPGQSPKLLIYYASNRYTGVPDRFTGSGSGTDFTFTISSV | Light chain, Human |
| | QVEDLTVYFCQQHYTSPLTFGAGTKLELKRTVAAPSVFIFPPSDEQLKS | Fc, mouse Fv |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 64 | MDWTWRFLFVVAAATGVQSEVQLQESGPELVKPGASVMISCKTSGYSFT | Anti-PSA Clone 10 |
| | DYNMNWVKQNNGKSLDWIGVISPDYGTPSYNQKFKGKATLTVDQSSSTA | Heavy chain, |
| | YMELNSLTSEDSAVYYCARGKGNYVVFAYWGQGTLVTVAAASTKGPSVF | Human Fc, mouse |
| | PLAPSSKSTSGGTAALGCLVKDYFPEPVTVSWNSGALTSGVHTFPAVLQ | Fv |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 65 | MRVPAQLLGLLLLWLSGARCSIVMTQTPKFLPVSAGDRVTMTCKASQSV | Anti-PSA Clone 56 |
| | VNNVVWYLQKPGQSPKLLIYYASNRYTGVPDRFTGSGSGTDFTFTISSV | Light chain, Human |
| | QVEDLAVYFCQQHYNSPLTFGAGTKLELKRTVAAPSVFIFPPSDEQLKS | Fc, mouse Fv |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 66 | MDWTWRFLFVVAAATGVQSEVQLQESGGGLVKPGGSLKLSCAASGFTFS | Anti-PSA Clone 56 |
| | TYAMSWVRQTPEKGLEWVAAISDSGTYTFYPDNVRGRFTISRDNAKNNL | Heavy chain, |
| | FLQMSHLKSEDTAMYYCTRDYYGSGSYWFFDVWGSGTTVTVASTKGPSV | Human Fc, mouse |
| | FPLAPSSKSTSGGTAALGCLVKDYFPEPVTVSWNSGALTSGVHTFPAVL | Fv |
| | QSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKC | |
| | KVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQ | |
| | GNVFSCSVMHEALHNHYTQKSLSLSPGK | |
| 67 | MRVPAQLLGLLLLWLSGARCDIVMTQAAFSNPVTLGTSASISCRSSKSL | Anti-PSA Clone 3 |
| | LHSDGITYLYWYLQRPGQSPQVLIYRMSNLASGVPDRFSGSGSGTDFTL | Light chain, Human |
| | RISRVEAEDVGVYYCAQMLEFPYTFGSGTKLEMKRTVAAPSVFIFPPSD | Fc, mouse Fv |
| | EQLKSGTASVVCLLNNFYPREAKVQWKVDNALQSGNSQESVTEQDSKDS | |
| | TYSLSSTLTLSKADYEKHKVYACEVTHQGLSSPVTKSFNRGEC | |
| 68 | MDWTWRFLFVVAAATGVQSEVQLQESGPVLVKPGASVKMSCKASGYTFT | Anti-PSA Clone 3 |
| | DYYMSWVKQSHGKSLEWIGVVNPYSGGSTYNQKFKGKATLTVDKSSTTA | Heavy chain, |
| | YLELNSLTSEDSAVYYCARGAEDNWGQGTTLTVSSASTKGPSVFPLAPS | Human Fc, mouse |
| | SKSTSGGTAALGCLVKDYFPEPVTVSWNSGALTSGVHTFPAVLQSSGLY | Fv |
| | SLSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFSC | |
| | SVMHEALHNHYTQKSLSLSPGK | |
| 69 | MRVPAQLLGLLLLWLSGARCDIQMTQSPSSLSASVGDRVTITCKASQSV | PSA1 Lc, Human |
| | VNNVVWYQQKPGKAPKLLIYYASNRYSGVPSRFSGSGSGTDFTLTISSL | Fc, humanized Fv |
| | QPEDFATYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 70 | MDWTWRFLFVVAAATGVQSQVQLVQSGAEVKKPGSSVKVSCKASGYSFT | PSA1 Hc, Human |
| | DYNMNWVRQAPGQGLEWIGVISPDYGTPSYAQKFQGRVTITADESTSTA | Fc, humanized Fv |
| | YMELSSLRSEDTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 71 | MRVPAQLLGLLLLWLSGARCDIVMTQTPLSLSVTPGQPASISCKASQSV | PSA2 Lc, Human |
| | VNNVVWYLQKPGQPPQLLIYYASNRYSGVPDRFSGSGSGTDFTLKISRV | Fc, humanized Fv |
| | EAEDVGVYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 72 | MDWTWRFLFVVAAATGVQSQVQLVQSGAEVKKPGSSVKVSCKASGYSFT | PSA2 Hc, Human |
| | DYNMNWVRQAPGQGLEWIGVISPDYGTPSYAQKFQGRVTITADESTSTA | Fc, humanized Fv |
| | YMELSSLRSEDTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 73 | MRVPAQLLGLLLLWLSGARCDIVMTQSPDSLAVSLGERATINCKASQSV | PSA3 Lc, Human |
| | VNNVVWYQQKPGQPPKLLIYYASNRYSGVPDRFSGSGSGTDFTLTISSL | Fc, humanized Fv |
| | QAEDVAVYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 74 | MDWTWRFLFVVAAATGVQSEVQLVQSGAEVKKPGATVKISCKVSGYSFT | PSA3 Hc, Human |
| | DYNMNWVQQAPGKGLEWIGVISPDYGTPSYAEKFQGRVTITADTSTDTA | Fc, humanized Fv |
| | YMELSSLRSEDTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 75 | MRVPAQLLGLLLLWLSGARCDIQMTQSPSSLSASVGDRVTITCKASQSV | PSA4 Lc, Human |
| | VNNVVWYQQKPGKAPKLLIYYASNRYSGVPSRFSGSGSGTEFTLTISSL | Fc, humanized Fv |
| | QPEDFATYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 76 | MDWTWRFLFVVAAATGVQSQVQLQESGPGLVKPSETLSLTCTVSGYSFT | PSA4 Hc, Human |
| | DYNMNWIRQPPGKGLEWIGVISPDYGTPSYNPSLKSRVTISVDTSKNQF | Fc, humanized Fv |
| | SLKLSSVTAADTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 77 | MRVPAQLLGLLLLWLSGARCDIQMTQSPSSLSASVGDRVTITCKASQSV | PSA5 Lc, Human |
| | VNNVVWYQQKPGKAPKLLIYYASNRYSGVPSRFSGSGSGTEFTLTISSL | Fc, humanized Fv |
| | QPEDFATYYCQQHYTSPLTFGPGTKVDIKRTVAAPSVFIFPPSDEQLKS | |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 78 | MDWTWRFLFVVAAATGVQSEVQLVQSGAEVKKPGATVKISCKVSGYSFT | PSA5 Hc, Human |
| | DYNMNWVQQAPGKGLEWIGVISPDYGTPSYAEKFQGRVTITADTSTDTA | Fc, humanized Fv |
| | YMELSSLRSEDTAVYYCARGKGNYVVFAYWGQGTLVTVSSASTKGPSVF | |
| | PLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 79 | MGWSCIILFLVATATGVHCQVQLKQSGPGLVAPSQSLSITCTVSGFSLT | 492, heavy, Murine |
| | GYGVNWVRQSPGKGLEWLGMIWGDGRTDYKSALKSRLSITKDNSKSQVF | Fc, murine Fv |
| | LKMNSLQTDDTARYFCASDYYGSGSFAYWGQGTLVTVSAASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 80 | MGWSCIILFLVATATGVHSDVVMTQSHKFMSTSVGDRVSITCKASQDVS | 492, light, Murine |
| | TAVAWYQQKSGQSPKLLIHSASYRYTGVPDRFTGSGSGTDFTFTISSVQ | Fc, murine Fv |
| | AEDLAVYYCQQHYSIPLTFGAGTKLELKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 81 | MDWTWRFLFVVAAATGVQSEVQLQESGPVLVKPGASVKMSCKASGYTFT | Anti-PSA Clone 3 |
| | DYYMSWVKQSHGKSLEWIGVVNPYSGGSTYNQKFKGKATLTVDKSSTTA | Heavy chain, |
| | YLELNSLTSEDSAVYYCARGAEDNWGQGTTLTVSSASTKGPSVFPLAPS | Human Fc, mouse |
| | SKSTSGGTAALGCLVKDYFPEPVTVSWNSGALTSGVHTFPAVLQSSGLY | Fv |
| | SLSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFSC | |
| | SVMHEALHNHYTQKSLSLSPGK | |
| 82 | MRVPAQLLGLLLLWLSGARCDIVMTQAAFSNPVTLGTSASISCRSSKSL | Anti-PSA Clone 3 |
| | LHSDGITYLYWYLQRPGQSPQVLIYRMSNLASGVPDRFSGSGSGTDFTL | Light chain, Human |
| | RISRVEAEDVGVYYCAQMLEFPYTFGSGTKLEMKRTVAAPSVFIFPPSD | Fc, mouse Fv |
| | EQLKSGTASVVCLLNNFYPREAKVQWKVDNALQSGNSQESVTEQDSKDS | |
| | TYSLSSTLTLSKADYEKHKVYACEVTHQGLSSPVTKSFNRGEC | |
| 83 | MRVPAQLLGLLLLWLSGARCSIVMTQTPKILPVSAGDRVTMTCKASQSV | Anti-PSA Clone 9 |
| | VNNVVWYLQKPGQSPKLLIYYASNRYTGVPDRFTGSGSGTDFTFTISSV | Light chain, Human |
| | QVEDLTVYFCQQHYTSPLTFGAGTKLELKRTVAAPSVFIFPPSDEQLKS | Fc, mouse Fv |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 84 | MDWTWRFLFVVAAATGVQSEVQLQESGPELVKPGASVKISCTASGYSFT | Anti-PSA Clone 9 |
| | DYNMNWVKQNNGKSPEWIGVISPDYGTTSYNQNFKGRATLTVDQSSSTA | heavy chain, |
| | YMQLNSLTSEDSAVYYCARGKGNYVVFAFWGQGTLVTVSAASTKGPSVF | Human Fc, mouse |
| | PLAPSSKSTSGGTAALGCLVKDYFPEPVTVSWNSGALTSGVHTFPAVLQ | Fv |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 85 | MRVPAQLLGLLLLWLSGARCSIVMTQTPKILPVSAGDRVTMTCKASQSV | Anti-PSA Clone 10 |
| | VNNVVWYLQKPGQSPKLLIYYASNRYTGVPDRFTGSGSGTDFTFTISSV | Light chain, Human |
| | QVEDLTVYFCQQHYTSPLTFGAGTKLELKRTVAAPSVFIFPPSDEQLKS | Fc, mouse Fv |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 86 | MDWTWRFLFVVAAATGVQSEVQLQESGPELVKPGASVMISCKTSGYSFT | Anti-PSA Clone 10 |
| | DYNMNWVKQNNGKSLDWIGVISPDYGTPSYNQKFKGKATLTVDQSSSTA | Heavy chain, |
| | YMELNSLTSEDSAVYYCARGKGNYVVFAYWGQGTLVTVAAASTKGPSVF | Human Fc, mouse |
| | PLAPSSKSTSGGTAALGCLVKDYFPEPVTVSWNSGALTSGVHTFPAVLQ | Fv |
| | SSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQG | |
| | NVFSCSVMHEALHNHYTQKSLSLSPGK | |
| 87 | MRVPAQLLGLLLLWLSGARCSIVMTQTPKFLPVSAGDRVTMTCKASQSV | Anti-PSA Clone 56 |
| | VNNVVWYLQKPGQSPKLLIYYASNRYTGVPDRFTGSGSGTDFTFTISSV | Light chain, Human |
| | QVEDLAVYFCQQHYNSPLTFGAGTKLELKRTVAAPSVFIFPPSDEQLKS | Fc, mouse Fv |
| | GTASVVCLLNNFYPREAKVQWKVDNALQSGNSQESVTEQDSKDSTYSLS | |
| | STLTLSKADYEKHKVYACEVTHQGLSSPVTKSFNRGEC | |
| 88 | MDWTWRFLFVVAAATGVQSEVQLQESGGGLVKPGGSLKLSCAASGFTFS | Anti-PSA Clone 56 |
| | TYAMSWVRQTPEKGLEWVAAISDSGTYTFYPDNVRGRFTISRDNAKNNL | Heavy chain, |
| | FLQMSHLKSEDTAMYYCTRDYYGSGSYWFFDVWGSGTTVTVASTKGPSV | Human Fc, mouse |
| | FPLAPSSKSTSGGTAALGCLVKDYFPEPVTVSWNSGALTSGVHTFPAVL | Fv |
| | QSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKC | |
| | KVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQ | |
| | GNVFSCSVMHEALHNHYTQKSLSLSPGK | |
| 89 | MGWSCIILFLVATATGVHCEVQLVQSGAEVKKPGATVKISCKVSGYTFS | Lysozyme Design 1 |
| | TYWMEWVQQAPGKGLEWMGEVLPGSGSTYYAEKFQGRVTITADTSTDTA | IgG1 heavy chain |
| | YMELSSLRSEDTAVYYCARGDGYFQHWGQGTLVTVSSASTKGPSVFPLA | |
| | PSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCP | |
| | PCPAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVF | |
| | SCSVLHEALHSHYTQKSLSLSPGK | |
| 90 | MGWSCIILFLVATATGVHSDIQMTQSPSSLSASVGDRVTITCKASQDVS | K1a, Human Fc, |
| | TAVAWFQQKPGKAPKLLIHSASYRVTGVPSRFSGSGSGTDFTLTISSLQ | humanized Fv |
| | PEDFATYYCQQHYSIPFTFGPGTKVDIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSENRGEC | |
| 91 | MGWSCIILFLVATATGVHCQITLKESGPTLVKPTQTLTLTCTVSGFSLT | H2a, Human Fc, |
| | GYGVNWIRQPPGKALEWLGMIWGDGRTDYSPSLKSRLTITKDTSKNQVV | humanized Fv |
| | LTMTNMDPVDTATYYCASDYYGSGSFAYWGQGTTVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 92 | MGWSCIILFLVATATGVHCQITLKESGPTLVKPTQTLTLTCTVSGFSLT | H2b, Human Fc, |
| | GYGVNWIRQPPGKALEWLGMIWGDGRTDYSPSLKSRLTITKDTSKNQVV | humanized Fv |
| | LTMTNMDPVDTATYYCASDYYGSGSFAYWGRGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 93 | MGWSCIILFLVATATGVHCQVQLVESGGGLVQPGGSLRLSCSVSGFSLT | H3, Human Fc, |
| | GYGVNWVRQAPGKGLEWLGMIWGDGRTDYADSVKGRFTISRDNSKNTLY | humanized Fv |
| | LQMSSLRAEDTAVYYCASDYYGSGSFAYWGQGTLVTVSSASTKGPSVFP | |
| | LAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGN | |
| | VFSCSVLHEALHSHYTQKSLSLSPGK | |
| 94 | MGWSCIILFLVATATGVHSDIQMTQSPSSLSASVGDRVTITCKASQDVS | K1a, Human Fc, |
| | TAVAWYQQKPGKAPKLLIHSASYRYTGVPSRFSGSGSGTDFTFTISSLQ | humanized Fv |
| | PEDIATYYCQQHYSIPLTFGQGTKVEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 95 | MGWSCIILFLVATATGVHSDIVMTQTPLSLSVTPGQPASISCKASQDVS | K2, Human Fc, |
| | TAVAWYLQKPGQPPQLLIHSASYRYTGVPDRFSGSGSGTDFTLKISRVE | humanized Fv |
| | AEDVGVYYCQQHYSIPLTFGPGTKVDIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 96 | MGWSCIILFLVATATGVHSEIVMTQSPATLSLSPGERATLSCKASQDVS | K3, Human Fc, |
| | TAVAWYQQKPGQAPRLLIHSASYRYTGIPARFSGSGSGTDFTLTISSLQ | humanized Fv |
| | PEDFAVYYCQQHYSIPLTFGQGTKVEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 97 | MGWSCIILFLVATATGVHSDIVMTQSPDSLAVSLGERATINCKASQDVS | K4, Human Fc, |
| | TAVAWYQQKPGQPPKLLIHSASYRYTGVPDRFSGSGSGTDFTLTISSLQ | humanized Fv |
| | AEDVAVYYCQQHYSIPLTFGQGTRLEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 98 | MGWSCIILFLVATATGVHSAIQLTQSPSSLSASVGDRVTITCKASQDVS | K1b, Human Fc, |
| | TAVAWYQQKPGKAPKLLIHSASYRYTGVPSRFSGSGSGTDFTLTISSLQ | humanized Fv |
| | PEDFATYYCQQHYSIPLTFGQGTKVEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 99 | MGWSCIILFLVATATGVHSDIVMTQSPDSLAVSLGERATINCKASQDVS | K4, Human Fc, |
| | TAVAWYQQKPGQPPKLLIHSASYRVTGVPDRFSGSGSGTDFTLTISSLQ | humanized Fv |
| | AEDVAVYYCQQHYSIPFTFGQGTRLEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 100 | Asn-Gly | Motif which is |
| | | excluded from |
| | | humanized designs |
| 101 | Asn-X-Ser/Thr | Motif which is |
| | | excluded from |
| | | humanized designs |
| 102 | DIELTQSPATLSVTPGDSVSLSCRASQSISNNLHWYQQKSHESPRLLIK | D44.1 light |
| | YVSQSSSGIPSRFSGSGSGTDFTLSINSVETEDFGMYFCQQSNSWPRTF | variable |
| | GGGTKLEIK | |
| 103 | QVQLQESGAEVMKPGASVKISCKATGYTFSTYWIEWVKQRPGHGLEWIG | D44.1 heavy |
| | EILPGSGSTYYNEKFKGKATFTADTSSNTAYMQLSSLTSEDSAVYYCAR | variable |
| | GDGNYGYWGQGTTLTVSSAS | |
| 104 | MGWSCIILFLVATATGVHSDIELTQSPATLSVTPGDSVSLSCRASQSIS | D44.1 Mouse Fab, |
| | NNLHWYQQKSHESPRLLIKYVSQSSSGIPSRFSGSGSGTDFTLSINSVE | light |
| | TEDFGMYFCQQSNSWPRTFGGGTKLEIKRTVAAPSVFIFPPSDEQLKSG | |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |
| 105 | MGWSCIILFLVATATGVHCQVQLQESGAEVMKPGASVKISCKATGYTFS | D44.1 Mouse Fab, |
| | TYWIEWVKQRPGHGLEWIGEILPGSGSTYYNEKFKGKATFTADTSSNTA | heavy |
| | YMQLSSLTSEDSAVYYCARGDGNYGYWGQGTTLTVSSASTTPPSVYPLA | |
| | PGSAAQTNSMVTLGCLVKGYFPEPVTVTWNSGSLSSGVHTFPAVLQSDL | |
| | YTLSSSVTVPSSTWPSQTVTCNVAHPASSTKVDKKIVPRDCGKHHHHHH | |
| 106 | MGWSCIILFLVATATGVHCEVQLVQSGAEVKKPGATVKISCKVSGYTFS | Anti-HEWL Design |
| | TYWMEWVQQAPGKGLEWMGEVLPGSGSTYYAEKFQGRVTITADTSTDTA | 6 IgG1, Human Fc, |
| | YMELSSLRSEDTAVYYCARGDGYFQHWGQGTLVTVSSASTKGPSVFPLA | humanized Fv, |
| | PSSKSTSGGTAALGCLVKDYFPEPVTVSWNSGALTSGVHTFPAVLQSSG | heavy |
| | LYSLSSVVTVPSSSLGTQTYICNVNHKPSNTKVDKRVEPKSCDKTHTCP | |
| | PCPAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVF | |
| | SCSVLHEALHSHYTQKSLSLSPGK | |
| 107 | MGWSCIILFLVATATGVHSEIVLTQSPDFQSVTPKEKVTITCRASQSIS | Anti-HEWL Design |
| | SNLHWYQQKPDQSPKLLIKYASQSFSGVPSRFSGSGSGTDFTLTINSLE | 6 IgG1, Human Fc, |
| | AEDAATYYCHQSNSWPRTFGPGTKVDIKRTVAAPSVFIFPPSDEQLKSG | humanized Fv, light |
| | TASVVCLLNNFYPREAKVQWKVDNALQSGNSQESVTEQDSKDSTYSLSS | |
| | TLTLSKADYEKHKVYACEVTHQGLSSPVTKSFNRGEC | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for preparing an antibody having an affinity to an antigen of interest, the method comprising:
i) providing a structural model of a first antibody having an affinity to the antigen of interest;
ii) generating all combinations of antibody segments derived from a plurality of human antibody germline sequences, and replacing corresponding amino acid residues in each of said combinations with amino acid residues of at least one complementarity determining region (CDR) of said first antibody, to thereby obtain a library of grafted human antibody sequences;
iii) threading each of said grafted human antibody sequences on said structural model to thereby obtain a plurality of threaded grafted human antibody structures, and subjecting each of said threaded grafted human antibody structures to constrained energy minimization to thereby obtain a plurality of relaxed grafted human antibody structures;
iv) ranking said plurality of relaxed grafted human antibody structures by an energy score; and
v) producing a second antibody sequence based on its ranking by energy score;
thereby obtaining an antibody having an affinity to an antigen of interest.

2. The method of claim 1, further comprising, prior to said threading, subjecting said structural model to energy minimization.

3. The method of claim 1, wherein said antibody segments are selected from the group consisting of heavy chain variable (V) gene segment, light chain variable (V) gene segment, heavy chain joining (J) gene segment, light chain joining (J) gene segment, kappa gene segment, and lambda gene segment.

4. The method of claim 1, further comprising removing sequences that exhibit more than two cysteines outside the at least one CDR from said library of grafted human antibody sequences.

5. The method of claim 1, further comprising removing sequences that exhibit Asn-Gly or Asn-X-Ser/Thr (where X is not Pro) motifs from said library of grafted human antibody sequences.

6. The method of claim 1, further comprising removing from said plurality of relaxed grafted human antibody structures a structure exhibiting more than 0.5 Å RMSD in a backbone atom of said at least one CDR compared to said structural model of a first antibody.

7. The method of claim 1, wherein said plurality of human antibody germline sequences is obtainable from a human genetics database.

8. The method of claim 7, wherein said human genetics database is the immunogenetics and immunoinformatics IMGT database.

9. The method of claim 1, wherein said first antibody is a non-human antibody.

10. The method of claim 9, wherein said method is a method of humanizing a non-human antibody.

11. The method of claim 1, wherein said first antibody is a human antibody and said method is a method of improving antibody stability.

12. The method of claim 1, further comprising after step (v) clustering said plurality of relaxed grafted human antibody structures according to V/J gene families to thereby obtain an energy ranked and gene family clustered library of humanized antibody designs and wherein said producing comprises producing at least two second antibody sequences based on their energy score and wherein at least two of said second antibody sequences are from different gene family clusters.

13. The method of claim 1, further comprising producing a protein expression vector comprising said produced second antibody sequence.

14. The method of claim 13, further comprising expressing a second antibody from said protein expression vector, testing binding of said second antibody to said antigen of interest and selecting said second antibody based on at least one of: the level of expression of said second antibody, binding of said second antibody to said antigen of interest and stability of said second antibody.

15. The method of claim 13, further comprising producing a plurality of protein expression vectors wherein each protein expression vector of said plurality comprises a different second antibody sequence.

16. The method of claim 15, further comprising expressing a second antibody from each protein expression vector of said plurality, testing binding of each second antibody to said antigen of interest and selecting a second antibody based on at least one of: the level of expression of said second antibody, binding of said second antibody to said antigen of interest and stability of said second antibody.

17. The method of claim 16, comprising selecting the second antibody with the best combination of expression level, stability and binding affinity to said antigen.

18. The method of claim 1, comprising identifying in said structural model amino acid residues of at least one CDR and replacing corresponding amino acid residues in each of said combinations with said identified amino acid residues.

19. The method of claim 1, wherein said at least one CDR is all CDRs that directly contact said antigen.

20. The method of claim 19, comprising identifying in said structural model amino acid residues of all CDRs that directly contact said antigen and replacing corresponding amino acid residues in each of said combinations with said identified amino acid residues.
